(19) United States
(12) Patent Application Publication
(10) Pub. No.: US 2024/0156778 A1
(43) Pub. Date: May 16, 2024

(54) THIOPHENE DERIVATIVES FOR USE IN TREATING PORTAL INFLAMMATION AND FIBROSIS

(71) Applicant: ENYO PHARMA, LYON (FR)

(72) Inventors: RAPHAËL DARTEIL, LYON (FR); ERIC MELDRUM, RIEHEN (CH); JACKY VONDERSCHER, SENOUILLAC (FR)

(21) Appl. No.: 17/769,344

(22) PCT Filed: Oct. 16, 2020
(86) PCT No.: PCT/EP2020/079147

§ 371 Date: Apr. 15, 2022

(30) Foreign Application Priority Data
Oct. 17, 2019 (EP) 19306353.4

(51) Int. Cl. A61K 31/381; A61K 31/575; A61P 1/16

(52) U.S. Cl. CPC A61K 31/381; A61K 31/575; A61P 1/16

(57) ABSTRACT

The present invention relates to a compound of formula (I) for use in a method for treating hepatic portal/periportal inflammation, optionally hepatic portal/periportal fibrosis.

### FIELD OF THE INVENTION

The present invention relates to the field of the medicine, in particular of liver diseases.

### BACKGROUND OF THE INVENTION

Some liver diseases have a specific aspect associated with hepatic periportal inflammation, sometimes with hepatic periportal fibrosis.

In this context, it could be interesting to consider Non-alcoholic steatohepatitis (NASH). The typical adult NASH histological pattern (termed NASH type 1) is characterized by the presence of steatosis (mainly macrovesicular) with ballooning degeneration and/or perisinusoidal fibrosis (zone 3 lobular involvement) and with the portal tracts being relatively spared. However, the pediatric type NASH (NASH Type 2) is described as the presence of steatosis along with portal inflammation and/or fibrosis in the absence of ballooning degeneration and perisinusoidal fibrosis.

More generally, Angulo et al (2015, Gastroenterology, 149, 389-397) reported that portal inflammation grade is inversely correlated with survival free of liver transplantation and survival free of liver-related events, whereas no correlation has been observed with steatosis grade and lobular inflammation grade. In addition, Gadd et al (2014, Hepatology, 59, 1393) reported that portal inflammation in NASH is strongly correlated with disease severity. Accordingly, NASH patients with portal inflammation represent a population with higher risk that need specific therapeutic care.

Obeticholic acid (OCA) has successfully completed phase III clinical trials for the treatment of NASH. While this drug has a significant effect on steatosis and lobular inflammation, it has no effect on the portal inflammation.

While WO2019/010024 discloses the use of cysteamine salt or cystamine salt for the treatment of patients suffering from a disease or disorder associated with periportal/portal liver inflammation, there remains a strong need for drugs capable of specifically acting on hepatic periportal inflammation and optionally on portal fibrosis.

### SUMMARY OF THE INVENTION

The present application provides compounds capable of specifically acting on hepatic periportal/portal inflammation and optionally on periportal/portal fibrosis. This effect is unexpected. Indeed, the inventors have also tested another compound of reference, namely Obeticholic acid (OCA). OCA has no effect on the hepatic portal/periportal inflammation at all. As OCA selectively inhibits NK-kB-mediated hepatic inflammatory response and suppresses NF-kB activation and that the compounds of the present invention also are able to inhibit NF-kB pathway, it is clear that the specific effect of the compounds of the present invention on the hepatic portal/periportal inflammation in comparison to the absence of effect of OCA cannot be solely based on this mechanism. Therefore, this specific pharmacological effect is surprising and unexpected.

This novel pharmacological effect supports the use of these compounds for treating diseases and disorders associated with a hepatic portal/periportal inflammation such as pediatric NAFLD and pediatric NASH.

In addition, this novel pharmacological effect supports the use of these compounds for diseases and disorders that present hepatic portal/periportal inflammation. In particular, the compounds of the invention are particularly useful for treating a subgroup of patients suffering from one of these diseases or disorders, said patients having in addition a hepatic portal/periportal inflammation.

For instance, as discussed in the background section, patients with NASH and having a periportal or portal inflammation are a subgroup of patients associated with a more severe disease. In other words, not all patients suffering from NASH have a periportal or portal inflammation. It has been shown that periportal/portal inflammation is inversely correlated with survival free of liver transplantation and survival free of liver-related events. Therefore, there is an interest to decrease periportal/portal inflammation in these patients and the NASH patients having a periportal or portal inflammation are a defined subgroup of patients having a greater therapeutic benefit of a treatment with the compounds of the present invention.

Accordingly, the present invention relates to
a compound of formula (I) for use for treating a hepatic periportal/portal inflammation in a subject, or
the use of a compound of formula (I) for use for treating a hepatic periportal/portal inflammation in a subject, or
a pharmaceutical composition comprising a compound of formula (I) for use for treating a hepatic periportal/portal inflammation in a subject,
a method for treating a hepatic periportal/portal inflammation in a subject, comprising administering a therapeutic amount of a compound of formula (I) to the subject, thereby decreasing the hepatic periportal/portal inflammation in the subject,

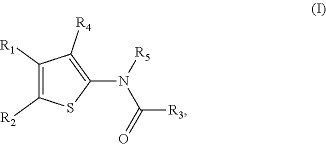

wherein:
R₁ represents a fused arylcycloalkyl or a cycloalkyl optionally substituted by at least one radical selected in the group consisting of:
   a halogen,
   a (C₁-C₆)alkyl or a (C₁-C₆)alkyloxy optionally substituted by at least one halogen, preferably optionally substituted by at least one fluorine, and
   a hydroxy,
   a —CO—R₆ or a —CO₂R₆ with R₆ being a hydrogen or a (C₁-C₆)alkyl, and an optionally substituted aryl;
R₂ represents:
   a hydrogen,
   a halogen,
   a (C₁-C₆)alkyl optionally substituted by at least one halogen, preferably optionally substituted by at least one fluorine,
   an optionally substituted aryl, or
   an optionally substituted cycloalkyl;
R₃ represents:
   a 5-10 membered ring, saturated or unsaturated selected in the group consisting of:
      an aryl optionally fused to a heterocycloalkyl, preferably selected from the group consisting of a dioxole, a morpholine, a dioxane, a tetrahydropyran, and a tetrahydrofuran,
      a heteroaryl,
      a cycloalkyl,
      a heterocycloalkyl, and
      a 5-10 membered bridged carbocyclyl or heterocyclyl,
      said 5-10 membered ring is optionally substituted by at least one radical selected in the group consisting of:
         a halogen,
         a (C₁-C₆)alkyl optionally substituted by at least one halogen, preferably optionally substituted by at least one fluorine, or by an optionally bridged heterocycloalkyl optionally substituted by a (C₁-C₆)alkyl,
         a —NH-(C₁-C₆)alkyl or a —N—((C₁-C₆)alkyl)₂, optionally substituted by at least one radical selected in the group consisting of a heterocycloalkyl, a cycloalkyl, a hydroxyl, a thiacycloalkyl-1,1dioxide and a (C₁-C₆)alkyloxy,
         a —NH-heterocycloalkyl, a —NH-cycloalkyl, a —N((C₁-C₆)alkyl)-heterocycloalkyl, or a —NH((C₁-C₆)alkyl)-thiacycloalkyl-1,1dioxide, optionally substituted by a hydroxyl, a (C₁-C₆)alkyl, a (C₁-C₆)alkyloxy or a —CO—R₆ with R₆ being a hydrogen or a (C₁-C₆)alkyl,
         a hydroxy, a —CN, a —CO—R₆ or a —CO₂R₆ with R₆ being a hydrogen or a (C₁-C₆)alkyl,
         a (Cl-C₆)alkyloxy optionally substituted by at least one radical selected in the group consisting of a halogen, preferably a fluorine, a hydroxy, a (C₁-C₆)alkyloxy, a —NR₂R₈ with R₇ and R₆ are independently a hydrogen or a (C₁-C₆)alkyl, a —NHCOR₉, a —NHCO₂R₉, with R₉ being a (C₁-C₆)alkyl, a —CO₂R₆ with R₆ being a hydrogen or a (C₁-C₆)alkyl, and a heterocycle,
         a —NHCOR₉, a —NHCO₂R₉, or a —SO₂R₉, with R₉ being a (C₁-C₆)alkyl, and
         a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
   a (C₁-C₆)alkyl or a (C₂-C₆)alkenyl, optionally substituted by a 5-10 membered ring as defined above or a —CO₂R₆ with R₆ being a hydrogen or a (C₁-C₆)alkyl;
R₄ represents a —COOH;
R₅ represents:
   a hydrogen, or
   a (C₁-C₆)alkyl optionally substituted by at least one halogen, preferably optionally substituted by at least one fluorine;
      and the stereoisomers, and the pharmaceutical salts thereof.

In one aspect, R₁ represents an optionally substituted fused arylcycloalkyl. Preferably, R₁ represents an optionally substituted fused arylcycloalkyl selected in a group consisting of an indanyl, a 1,2,3,4-tetrahydronaphtalenyl, and a 6,7,8,9-tetrahydro-5H-benzo[7]annulenyl, preferably an indanyl and a 1,2,3,4-tetrahydronaphtalenyl, more preferably a 1,2,3,4-tetrahydronaphtalenyl, preferably being

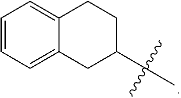 [SMILES: *C1CCc2ccccc2C1]

Optionally, R₃ represents an aryl optionally fused to a heterocycloalkyl, preferably selected from the group consisting of a dioxole, a morpholine, a dioxane, a tetrahydropyran, and a tetrahydrofuran, or a heteroaryl, said aryl, fused aryl, or heteroaryl is optionally substituted by at least one radical selected in the group consisting of:
a heterocycloalkyl or a bridged heterocycloalkyl, optionally substituted by a (C₁-C₆)alkyl, a (C₁-C₆)alkyloxy, or a ketone,
a thiaheterocycloalkyl-1,1-dioxide, a heterocycloalkyloxy, or a cycloalkyloxy;
(C₁-C₆)alkyloxy or a (C₁-C₆)alkyl, optionally substituted by at least one halogen, preferably a fluorine, or a (Cl-C₆)alkyloxy,
a halogen, preferably a fluorine or a chlorine,
a —NH—(C₁-C₆)alkyl or a —N—((C₁-C₆)alkyl)₂, optionally substituted by a heterocycloalkyl, a cycloalkyl, a hydroxyl, a thiacycloalkyl-1,1-dioxide or a (C₁-C₆)alkyloxy,
a —NH-heterocycloalkyl, a —N((C₁-C₆)alkyl)-heterocycloalkyl, or a —NH((C₁-C₆)alkyl)-thiacycloalkyl-1,1-dioxide,
a hydroxy,
a —CN,
a (C₁-C₆)alkyl substituted by an optionally bridged heterocycloalkyl or an optionally substituted heterocycloalkyl; and
a —SO₂R₉, with R₉ being a (C₁-C₆)alkyl.

More specifically, R₃ represents a phenyl, a pyridinyl or a pyrimidinyl, preferably a phenyl, optionally substituted by at least one radical selected in the group consisting of:
a morpholinyl optionally substituted by at least one methyl,
a —NH-tetrahydropyranyl,
a —NH-(C₁-C₆)alkyl or a —N(CH₃)(C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy,
an azetidinyl optionally substituted by a (C₁-C₆)alkyloxy,
a pyrrolidin-2-one,
a 6-oxa-3-azabicyclo[3.1.1]heptane, or a 8 oxa-3-azabicyclo[3.2.1]octane,
a (C₁-C₆)alkyloxy, optionally substituted by at least one halogen, preferably a fluorine, or one (C₁-C₆)alkyloxy,
a halogen, preferably a fluorine and a chlorine,
a hydroxy,
a —CN,
a —SO₂—CH₃,
a 1,1-dioxo-1,2-thiazolidin,
a cyclobutyloxy, or a tetra hyd ropyra nyloxy,
a (C₁-C₆)alkyl optionally substituted by at least one halogen, preferably optionally substituted by at least one fluorine, and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

Optionally, R₂ represents
a hydrogen,
a halogen, preferably a chlorine or a fluorine, and
an optionally substituted (C₃-C₆)cycloalkyl, preferably cyclopropyl; preferably a hydrogen, a chlorine or a fluorine.

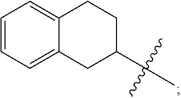 [SMILES: *C1CCc2ccccc2C1]

Optionally, said compound is such as R₁ is R₂ is a hydrogen or a halogen, preferably a halogen such as F or Cl; R₁ is a phenyl optionally substituted by a halogen such as F or Cl; and R₅ is a hydrogen.

In a particular aspect, said compound is selected in the group consisting of compounds of the table A.

In another particular aspect, said compound is selected in the group consisting of compounds of the table B.

Optionally, the subject has a hepatic portal/periportal inflammation of grade 1 (mild) or grade 2 (more than mild) as defined in the specification.

In a first aspect, the subject suffers from a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury, biliary atresia, idiopathic neonatal hepatitis syndrome, progressive familial intrahepatic cholestasis (PFIC) and primary and secondary mitochondrial hepatopathies.

Optionally, the subject suffers from a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), biliary atresia, idiopathic neonatal hepatitis syndrome, progressive familial intrahepatic cholestasis (PFIC), Reye's syndrome, Indian childhood cirrhosis, Idiopathic infantile copper toxicosis, Neonatal iron storage disease, Type I Tyrosinemia, and Zellweger syndrome.

In a very particular aspect, the disease is selected from the group consisting of pediatric non-alcoholic fatty liver disease (NAFLD) and pediatric non-alcoholic steatohepatitis (NASH). In particular, the subject is less than 18 years old.

In a second aspect, the subject has a hepatic portal/periportal inflammation and, optionally a hepatic portal/periportal fibrosis, and suffers from a disease selected from the group consisting of viral hepatitis, in particular chronic viral hepatitis, hemochromatosis and NASH.

Optionally, the compound is to be used in combination with another active ingredient, in particular another active ingredient which has a weak effect or no effect on hepatic portal/periportal inflammation. Optionally, the other active ingredient is Obeticholic acid (OCA).

The present invention further relates to a pharmaceutical composition comprising a compound as disclosed herein and Obeticholic acid (OCA). More specifically, the pharmaceutical composition comprising this combination is for use for treating a disease selected from the group of
pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury, biliary atresia, idiopathic neonatal hepatitis syndrome, progressive familial intrahepatic cholestasis (PFIC) and primary and secondary mitochondrial hepatopathies, preferably a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), biliary atresia, idiopathic neonatal hepatitis syndrome, progressive familial intrahepatic cholestasis (PFIC), Reye's syndrome, Indian childhood cirrhosis, Idiopathic infantile copper toxicosis, Neonatal iron storage disease, Type I Tyrosinemia, and Zellweger syndrome; and
a disease characterized by the occurrence of hepatic portal/periportal inflammation, optionally with a hepatic portal/periportal fibrosis, and selected from the group consisting of viral hepatitis, in particular chronic viral hepatitis, hemochromatosis and NASH.

The present invention further relates to a product or kit containing a compound as disclosed herein and Obeticholic acid (OCA) as a combined preparation for simultaneous, separate or sequential use, in particular in the treatment of a disease selected from the group of
pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury, biliary atresia, idiopathic neonatal hepatitis syndrome, progressive familial intrahepatic cholestasis (PFIC) and primary and secondary mitochondrial hepatopathies, preferably a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), biliary atresia, idiopathic neonatal hepatitis syndrome, progressive familial intrahepatic cholestasis (PFIC), Reye's syndrome, Indian childhood cirrhosis, Idiopathic infantile copper toxicosis, Neonatal iron storage disease, Type I Tyrosinemia, and Zellweger syndrome; and
a disease characterized by the occurrence of hepatic portal/periportal inflammation, optionally with a hepatic portal/periportal fibrosis, and selected from the group consisting of viral hepatitis, in particular chronic viral hepatitis, hemochromatosis and NASH.

BRIEF DESCRIPTION OF THE FRIGURES

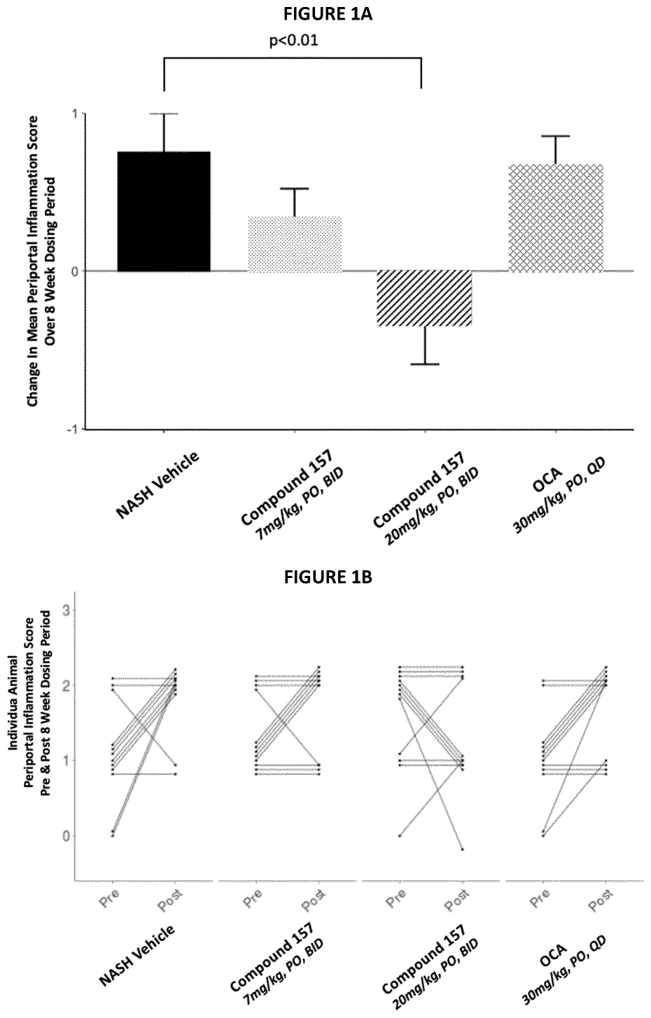

FIG. 1: Periportal inflammation. FIG. 1A: Change in Mean periportal inflammation score over 8 week dosing period. FIG. 1B: Individual animal periportal inflammation score pre and post 8 week dosing period.

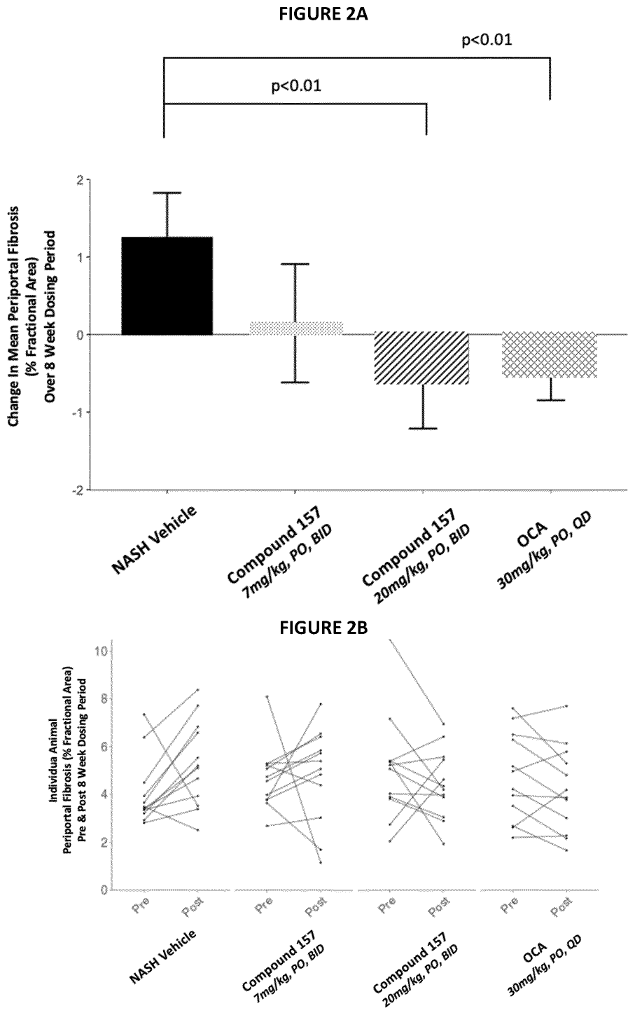

FIG. 2: Periportal fibrosis. FIG. 2A: Change in Mean periportal fibrosis (% of fractional area) over 8 week dosing period. FIG. 2B: Individual animal periportal fibrosis (% of fractional area) pre and post 8 week dosing period.

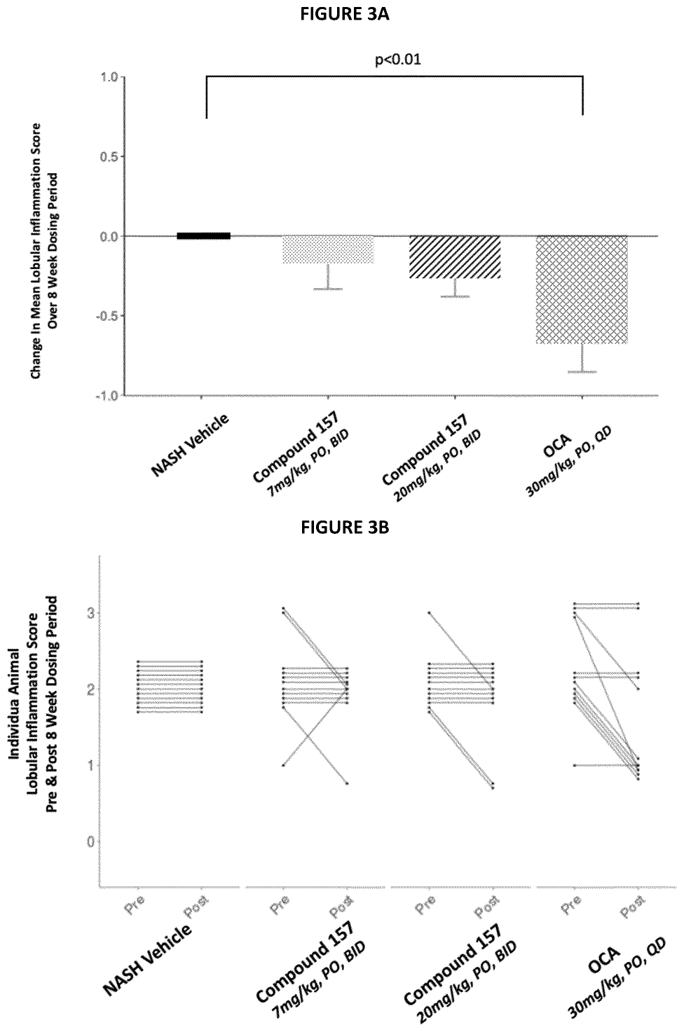

FIG. 3: Lobular inflammation. FIG. 3A: Change in Mean lobular inflammation score over 8 week dosing period. FIG. 3B: individual animal lobular inflammation score pre and post 8 week dosing period.

### DETAILED DESCRIPTION OF THE INVENTION

### Definitions

According to the present invention, the terms below have the following meanings:

The terms mentioned herein with prefixes such as for example C₁-C₃, C₁-C₆ or C₂-C₆ can also be used with lower numbers of carbon atoms such as C₁-C₂, C₁-C₅, or C₂-C₅. If, for example, the term C₁-C₃ is used, it means that the corresponding hydrocarbon chain may comprise from 1 to 3 carbon atoms, especially 1, 2 or 3 carbon atoms. If, for example, the term C₁-C₆ is used, it means that the corresponding hydrocarbon chain may comprise from Ito 6 carbon atoms, especially 1, 2, 3, 4, 5 or 6 carbon atoms. If, for example, the term C₂-C₆ is used, it means that the corresponding hydrocarbon chain may comprise from 2 to 6 carbon atoms, especially 2, 3, 4, 5 or 6 carbon atoms.

The term “alkyl” refers to a saturated, linear or branched aliphatic group. The term “(C₁-C₃)alkyl” more specifically means methyl, ethyl, propyl, or isopropyl. The term “(C₁-C₆)alkyl” more specifically means methyl, ethyl, propyl, isopropyl, butyl, isobutyl, tert-butyl, pentyl or hexyl. In a preferred embodiment, the “alkyl” is a methyl, an ethyl, a propyl, an isopropyl, or a tert-butyl, more preferably a methyl.

The term “alkenyl” refers to an unsaturated, linear or branched aliphatic group comprising at least one carbon-carbon double bound. The term “(C₂-C₆)alkenyl” more specifically means ethenyl, propenyl, isopropenyl, butenyl, isobutenyl, pentenyl, or hexenyl.

The term “alkoxy” or “alkyloxy” corresponds to the alkyl group as above defined bonded to the molecule by an —O— (ether) bond. (C₁-C₃)alkoxy includes methoxy, ethoxy, propyloxy, and isopropyloxy. (C₁-C₆)alkoxy includes methoxy, ethoxy, propyloxy, isopropyloxy, butyloxy, isobutyloxy, tert-butyloxy, pentyloxy and hexyloxy. In a preferred embodiment, the “alkoxy” or “alkyloxy” is a methoxy.

The term “cycloalkyl” corresponds to a saturated or unsaturated mono-, bi- or tri-cyclic alkyl group comprising between 3 and 20 atoms of carbons. It also includes fused, bridged, or spiro-connected cycloalkyl groups. The term “cycloalkyl” includes for instance cyclopropyl, cyclobutyl, cyclopentyl, and cyclohexyl. The term “cycloalkyl” may also refer to a 5-10 membered bridged carbocyclyl such as bicyclo[2,2,1]heptanyl, bicyclo[2,2,2]octanyl, bicyclo[1.1.1]pentanyl, or adamantyl, preferably bicyclo[2,2,1]heptanyl. In a preferred embodiment, the “cycloalkyl” is a cyclopropyl, cyclobutyl, cyclopentyl or a cyclohexyl.

The term “heterocycloalkyl” corresponds to a saturated or unsaturated cycloalkyl group as above defined further comprising at least one heteroatom such as nitrogen, oxygen, or sulphur atom. It also includes fused, bridged, or Spiro-connected heterocycloalkyl groups. Representative heterocycloalkyl groups include, but are not limited to 3-dioxolane, benzo [1,3] dioxolyl, azetidinyl, oxetanyl, pyrazolinyl, pyranyl, thiomorpholinyl, pyrazolidinyl, piperidyl, piperazinyl, 1,4-dioxanyl, imidazolinyl, pyrrolinyl, pyrrolidinyl, piperidinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranyl, tetrahydropyranyl, tetrahydrofuranyl, and tetrahydrothiophenyl. The term “heterocycloalkyl” may also refer to a 5-10 membered bridged heterocyclyl such as 7-oxabicyclo[2,2,1]heptanyl, 6-oxa-3-azabicyclo[3,1,1]heptanyl, and 8-oxa-3-azabicyclo[3,1,1]octanyl. In a particular embodiment, it may also refer to Spiro-connected heterocycloalkyl groups or spiroheterocycloalkyl groups such as for instance oxetanyl Spiro-connected with azetidinyl or piperidinyl. In a preferred embodiment, the heterocycloalkyl group is azetidinyl, oxetanyl, pyranyl, tetrahydrofuranyl, tetrahydropyranyl, morpholinyl, piperidinyl, piperazinyl, and oxetanyl Spiro-connected with azetidinyl or piperidinyl.

The term “aryl” corresponds to a mono- or bi-cyclic aromatic hydrocarbons having from 6 to 12 carbon atoms. For instance, the term “aryl” includes phenyl, biphenyl, or naphthyl. In a preferred embodiment, the aryl is a phenyl.

The term “heteroaryl” as used herein corresponds to an aromatic, mono- or poly-cyclic group comprising between 5 and 14 atoms and comprising at least one heteroatom such as nitrogen, oxygen or sulphur atom. Examples of such mono- and poly-cyclic heteroaryl group may be: pyridinyl, thiazolyl, thiophenyl, furanyl, pyrrolyl, pyrazolyl, imidazolyl, triazolyl, tetrazolyl, benzofuranyl, thianaphthalenyl, indolyl, indolinyl, quinolinyl, isoquinolinyl, benzimidazolyl, tetrahydroquinolinyl, tetrahydroisoquinolinyl, triazinyl, thianthrenyl, isobenzofuranyl, chromenyl, xanthenyl, phenoxanthinyl, isothiazolyl, isoxazolyl, pyrazinyl, pyridazinyl, indolizinyl, isoindolyl, indazolyl, purinyl, quinolizinyl, phtalazinyl, naphthyridinyl, quinoxalinyl, quinazolinyl, cinnolinyl, pteridinyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, indolinyl, isoindolinyl, oxazolidinyl, benzotriazolyl, benzoisoxazolyl, oxindolyl, benzoxazolinyl, benzothienyl, benzothiazolyl, isatinyl, dihydropyridyl, pyrimidinyl, s-triazinyl, oxazolyl, or thiofuranyl. In a preferred embodiment, the heteroaryl group is a pyridinyl, furanyl, pyrazolyl, pyrazinyl, pyrimidinyl, pyridazinyl, and isoxazolyl.

The terms “fused arylheterocycloalkyl” and “fused arylcycloalkyl” correspond to a bicyclic group in which an aryl as above defined is bounded to the heterocycloalkyl or the cycloalkyl as above defined by at least two carbons. In other terms, the aryl shares a carbon bond with the heterocycloalkyl or the cycloalkyl. A fused arylheterocycloalkyl is for instance a benzodioxole (phenyl fused to a dioxole), an isobenzofurane or a benzomorpholine (phenyl fused to a morpholine. A fused arylcycloalkyl is for instance an indanyl, a 1,2,3,4-tetrahydronaphtalenyl (also called tetralinyl), or a 6,7,8,9-tetrahydro-5H-benzo[7]annulenyl (fused phenyl-C₇-cycloalkyl). The term “fused bicycloalkyl” corresponds to a bicyclic group in which a cycloalkyl as above defined is bounded to the cycloalkyl as above defined by at least two carbons. A fused bicycloalkyl is for instance a bicyclo[4.1.0]heptanyl.

The term “halogen” corresponds to a fluorine, chlorine, bromine, or iodine atom, preferably a fluorine, chlorine or bromine.

The expression “substituted by at least” means that the radical is substituted by one or several groups of the list.

The expression “optionally substituted” means, without any otherwise precision, optionally substituted by a hydroxy, a halogen, a (C₁-C₆)alkyl optionally substituted by at least one halogen, preferably optionally substituted by at least one fluorine, or a (C₁-C₆)alkoxy optionally substituted by at least one halogen, preferably optionally substituted by at least one fluorine.

The “stereoisomers” are isomeric compounds that have the same molecular formula and sequence of bonded atoms, but differ in the 3D-dimensional orientations of their atoms in space.

The stereoisomers include enantiomers, diastereoisomers, Cis-trans and E-Z isomers, conformers, and anomers. In a preferred embodiment of the invention, the stereoisomers include diastereoisomers and enantiomers. The enantiomers compounds may be prepared from the racemate compound using any purification method known by a skilled person, such as LC/MS and chiral HPLC analysis methods and chiral SFC purification methods such as those disclosed in the examples (Example A—Chemistry, Table 1 and Table 3).

The “pharmaceutically salts” include inorganic as well as organic acids salts. Representative examples of suitable inorganic acids include hydrochloric, hydrobromic, hydroiodic, phosphoric, and the like. Representative examples of suitable organic acids include formic, acetic, trichloroacetic, trifluoroacetic, propionic, benzoic, cinnamic, citric, fumaric, maleic, methanesulfonic and the like. Further examples of pharmaceutically inorganic or organic acid addition salts include the pharmaceutically salts listed in J. Pharm. Sci. 1977, 66, 2, and in Handbook of Pharmaceutical Salts: Properties, Selection, and Use edited by P. Heinrich Stahl and Camille G. Wermuth 2002. In a preferred embodiment, the salt is selected from the group consisting of maleate, chlorhydrate, bromhydrate, and methanesulfonate. The “pharmaceutically salts” also include inorganic as well as organic base salts. Representative examples of suitable inorganic bases include sodium or potassium salt, an alkaline earth metal salt, such as a calcium or magnesium salt, or an ammonium salt. Representative examples of suitable salts with an organic base includes for instance a salt with methylamine, dimethylamine, trimethylamine, piperidine, morpholine or tris-(2-hydroxyethyl)amine. In a preferred embodiment, the salt is selected from the group consisting of sodium and potassium salt.

As used herein, the terms “treatment”, “treat” or “treating” refer to any act intended to ameliorate the health status of patients such as therapy, prevention, prophylaxis and retardation of a disease, in particular an infection, preferably a viral infection. In certain embodiments, such terms refer to the amelioration or eradication of the disease, or symptoms associated with it. In other embodiments, this term refers to minimizing the spread or worsening of the disease, resulting from the administration of one or more therapeutic agents to a subject with such a disease.

As used herein, the terms “subject”, “individual” or “patient” are interchangeable and refer to an animal, preferably to a mammal, even more preferably to a human, including adult, child, newborn and human at the prenatal stage. However, the term “subject” can also refer to non-human animals, in particular mammals such as dogs, cats, horses, cows, pigs, sheep and non-human primates, among others.

The terms “quantity,” “amount,” and “dose” are used interchangeably herein and may refer to an absolute quantification of a molecule.

As used herein, the terms “active principle”, “active ingredient” and “active pharmaceutical ingredient” are equivalent and refers to a component of a pharmaceutical composition having a therapeutic effect.

As used herein, the term “therapeutic effect” refers to an effect induced by an active ingredient, or a pharmaceutical composition according to the invention, capable to prevent or to delay the appearance or development of a disease or disorder, or to cure or to attenuate the effects of a disease or disorder.

As used herein, the term “effective amount” refers to a quantity of an active ingredient or of a pharmaceutical composition which prevents, removes or reduces the deleterious effects of the disease, particularly infectious disease. It is obvious that the quantity to be administered can be adapted by the man skilled in the art according to the subject to be treated, to the nature of the disease, etc. In particular, doses and regimen of administration may be function of the nature, of the stage and of the severity of the disease to be treated, as well as of the weight, the age and the global health of the subject to be treated, as well as of the judgment of the doctor.

As used herein, the term “excipient or pharmaceutically acceptable carrier” refers to any ingredient except active ingredients that is present in a pharmaceutical composition. Its addition may be aimed to confer a particular consistency or other physical or gustative properties to the final product. An excipient or pharmaceutically acceptable carrier must be devoid of any interaction, in particular chemical, with the active ingredients. Compounds

The compounds of the present invention can be any compound disclosed in WO 2019/154956 or WO 2019/154953, the disclosure of which being incorporated herein by reference.

In a first aspect, the compounds of the present invention can be any compound disclosed in WO 2019/154956, in particular any compound disclosed in Table A. In a second aspect, the compounds of the present invention can be any compound disclosed in WO 2019/154953, in particular any compound disclosed in Table A.

In a particular aspect, the compounds of the present invention have the formula (I)

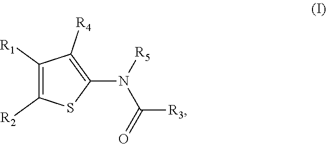

wherein:
R₁ represents a fused arylcycloalkyl or a cycloalkyl optionally substituted by at least one radical selected in the group consisting of:
   a halogen,
   a (C₁-C₆)alkyl or a (C₁-C₆)alkyloxy optionally substituted by at least one halogen, preferably optionally substituted by at least one fluorine, and
   a hydroxy,
   a —CO—R₆ or a —CO₂R₆ with R₆ being a hydrogen or a (C₁-C₆)alkyl, and
   an optionally substituted aryl;
R₂ represents:
   a hydrogen,
   a halogen,
   a (C₁-C₆)alkyl optionally substituted by at least one halogen, preferably optionally substituted by at least one fluorine,
   an optionally substituted aryl, or
   an optionally substituted cycloalkyl;
R₃ represents:
   a 5-10 membered ring, saturated or unsaturated selected in the group consisting of:
      an aryl optionally fused to a heterocycloalkyl, preferably selected from the group consisting of a dioxole, a morpholine, a dioxane, a tetrahydropyran, and a tetra hydrofuran,
      a heteroaryl,
      a cycloalkyl,
      a heterocycloalkyl, and
      a 5-10 membered bridged carbocyclyl or heterocyclyl, said 5-10 membered ring is optionally substituted by at least one radical selected in the group consisting of:
         a halogen,
         a (C₁-C₆)alkyl optionally substituted by at least one halogen, preferably optionally substituted by at least one fluorine, or by an optionally bridged heterocycloalkyl optionally substituted by a (C₁-C₆)alkyl,
         a —NH-(C₁-C₆)alkyl or a —N—((C₁-C₆)alkyl)₂, optionally substituted by at least one radical selected in the group consisting of a heterocycloalkyl, a cycloalkyl, a hydroxyl, a thiacycloalkyl-1,ldioxide and a (C₁-C₆)alkyloxy,
         a —NH-heterocycloalkyl, a —NH-cycloalkyl, a —N((C₁-C₆)alkyl)-heterocycloalkyl, or a —NH((C₁-C₆)alkyl)-thiacycloalkyl-1,1dioxide, optionally substituted by a hydroxyl, a (C₁-C₆)alkyl, a (C₁-C₆)alkyloxy or a —CO—R₆ with R₆ being a hydrogen or a (C₁-C₆)alkyl,
         a hydroxy, a —CN, a —CO—R₆ or a —CO₂R₆ with R₆ being a hydrogen or a (C₁-C₆)alkyl,
         a (Cl-C₆)alkyloxy optionally substituted by at least one radical selected in the group consisting of a halogen, preferably a fluorine, a hydroxy, a (C₁-C₆)alkyloxy, a —NR₂R₈ with R₇ and Rs are independently a hydrogen or a (C₁-C₆)alkyl, a —NHCOR₉, a —NHCO₂R₉, with R₉ being a (C₁-C₆)alkyl, a —CO₂R₆ with R₆ being a hydrogen or a (C₁-C₆)alkyl, and a heterocycle,
         a —NHCOR₉, a —NHCO₂R₉, or a —SO₂R₉, with R₉ being a (C₁-C₆)alkyl, and
         a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
   a (C₁-C₆)alkyl or a (C₂-C₆)alkenyl, optionally substituted by a 5-10 membered ring as defined above or a —CO₂R₆ with R₆ being a hydrogen or a (C₁-C₆)alkyl;
R₄ represents a —COOH;
R₅ represents:
   a hydrogen, or
   a (C₁-C₆)alkyl optionally substituted by at least one halogen, preferably optionally substituted by at least one fluorine; and the stereoisomers, and the pharmaceutical salts thereof.

Preferably, R₂ represents a hydrogen, a halogen, preferably a chlorine or a fluorine, and an optionally substituted (C₃-C₆)cycloalkyl, preferably cyclopropyl. In a preferred aspect, R₂ represents a hydrogen, a chlorine or a fluorine. In a specific aspect, R₂ is a hydrogen. In another specific aspect, R₂ is a chlorine or a fluorine.

Preferably, R₃ represents an aryl optionally fused to a heterocycloalkyl, preferably selected from the group consisting of a dioxole, a morpholine, a dioxane, a tetrahydropyran, and a tetrahydrofuran, or a heteroaryl, said aryl, fused aryl, or heteroaryl is optionally substituted by at least one radical selected in the group consisting of:
a heterocycloalkyl or a bridged heterocycloalkyl, optionally substituted by a (C₁-C₆)alkyl, a (C₁-C₆)alkyloxy, or a ketone,
a thiaheterocycloalkyl-1,1-dioxide, a heterocycloalkyloxy, or a cycloalkyloxy;
a (C₁-C₆)alkyloxy or a (C₁-C₆)alkyl, optionally substituted by at least one halogen, preferably a fluorine, or a (Cl-C₆)alkyloxy,
a halogen, preferably a fluorine or a chlorine,
a —NH-(C₁-C₆)alkyl or a —N—((C₁-C₆)alkyl)₂, optionally substituted by a heterocycloalkyl, a cycloalkyl, a hydroxyl, a thiacycloalkyl-1,1-dioxide or a (C₁-C₆)alkyloxy,
a —NH-heterocycloalkyl, a —N((C₁-C₆)alkyl)-heterocycloalkyl, or a —NH((C₁-C₆)alkyl)-thiacycloalkyl-1,1-dioxide,
a hydroxy,
a —CN,
a (C₁-C₆)alkyl substituted by an optionally bridged heterocycloalkyl or an optionally substituted heterocycloalkyl; and
a —SO₂R₉, with R₉ being a (C₁-C₆)alkyl.

More particularly, R₃ represents a phenyl, a pyridinyl or a pyrimidinyl, preferably a phenyl, optionally substituted by at least one radical selected in the group consisting of:
a morpholinyl optionally substituted by at least one methyl,
a —NH-tetrahydropyranyl,
a —NH-(C₁-C₆)alkyl or a —N(CH 3)(C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy,
an azetidinyl optionally substituted by a (Cl-C₆)alkyloxy,
a pyrrolidin-2-one,
a 6-oxa-3-azabicyclo[3.1.1]heptane, or a 8 oxa-3-azabicyclo[3.2.1]octane,
a (C₁-C₆)alkyloxy, optionally substituted by at least one halogen, preferably a fluorine, or one (C₁-C₆)alkyloxy,
a halogen, preferably a fluorine and a chlorine,
a hydroxy,
a —CN,
a —SO₂-CH 3,
a 1,1-dioxo-1,2-thiazolidin,
a cyclobutyloxy, or a tetrahydropyranyloxy,
a (C₁-C₆)alkyl optionally substituted by at least one halogen, preferably optionally substituted by at least one fluorine, and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

In a particular aspect, R₃ is a radical selected in the group consisting of:

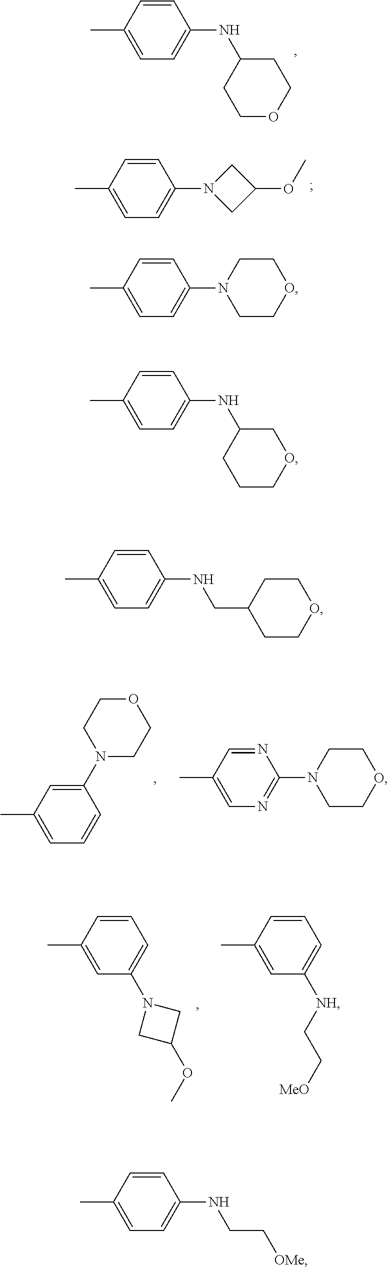

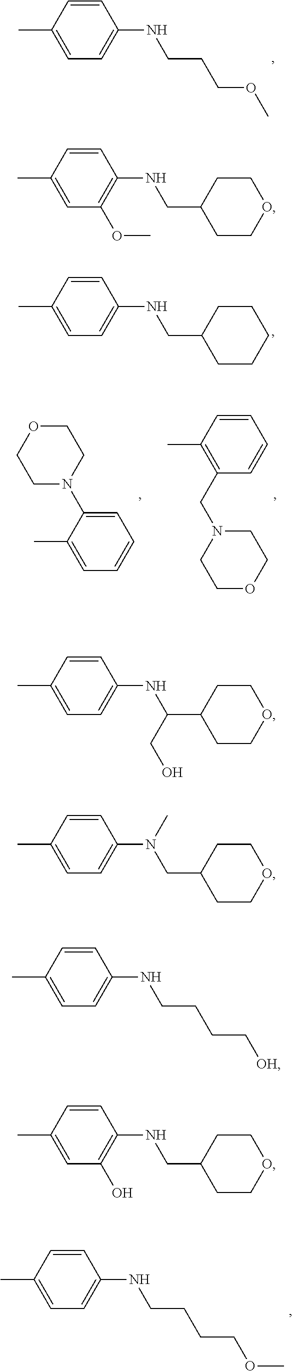

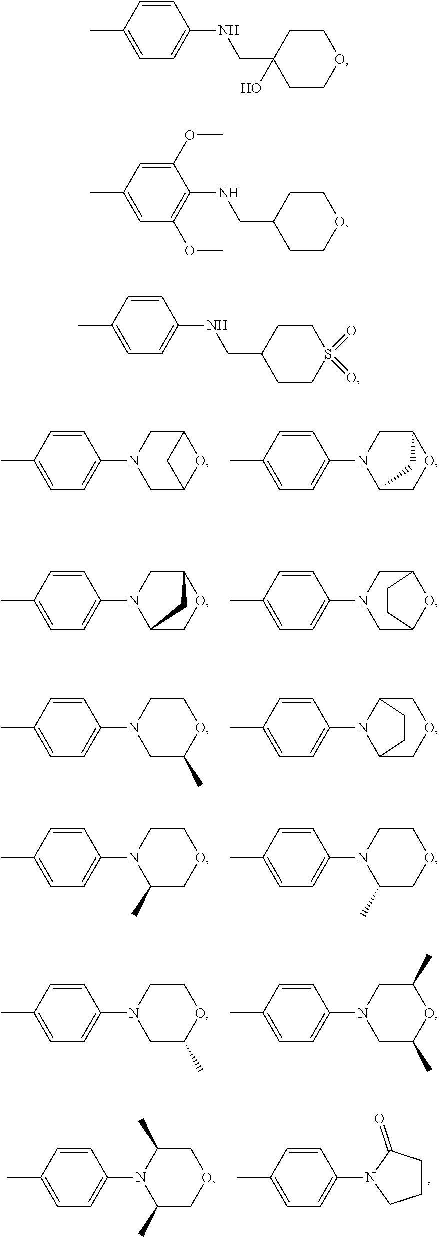

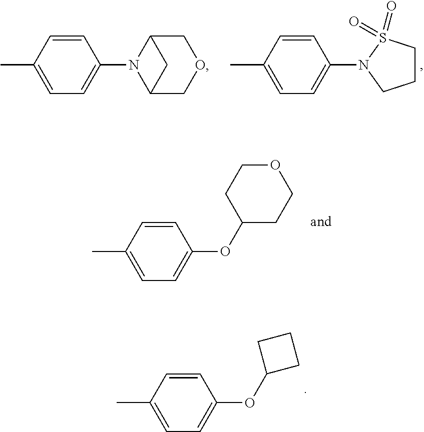

In another particular aspect, R₃ is a radical selected in the group consisting of:

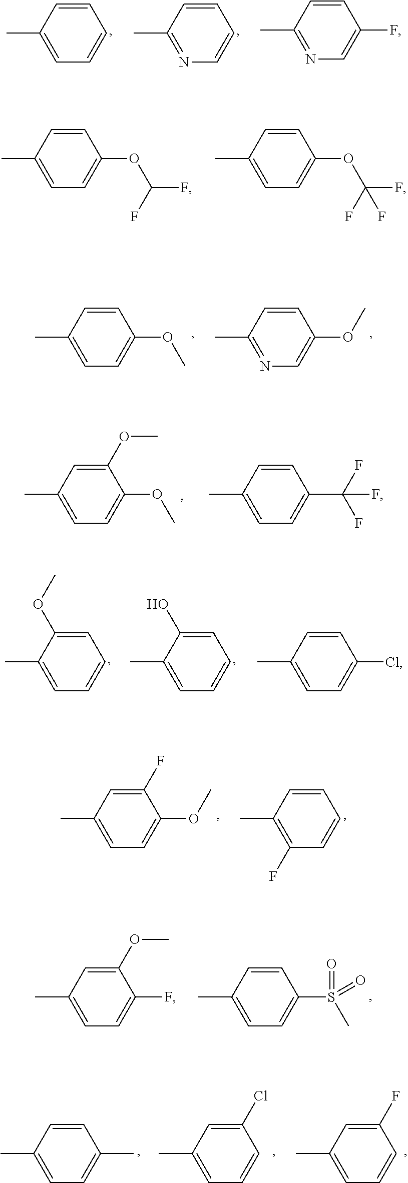

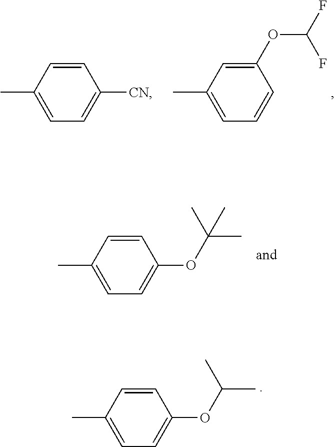

In a further particular aspect, R₃ is a radical selected in the group consisting of:

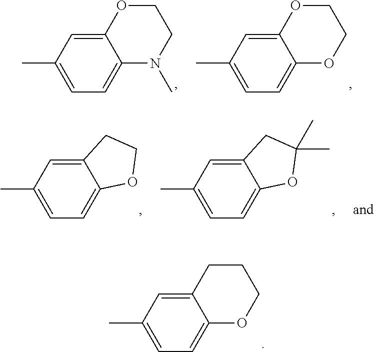

In a very specific aspect, R₃ represents a phenyl, optionally substituted by a halogen, preferably a fluorine. In this aspect, R₃ can be

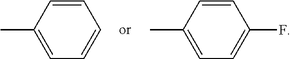

In a particular aspect, R₂ represents a halogen, preferably a fluorine or a chlorine, and R₃ can be

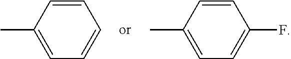

In the first aspect, R₁ represents an optionally substituted fused arylcycloalkyl. More specifically, R₁ represents an optionally substituted fused arylcycloalkyl selected in a group consisting of an indanyl, a 1,2,3,4-tetrahydronaphtalenyl, and a 6,7,8,9-tetrahydro-5H-benzo[7]annulenyl, preferably an indanyl and a 1,2,3,4-tetrahydronaphtalenyl, more preferably a 1,2,3,4-tetrahydronaphtalenyl.

For instance, the optionally substituted fused arylcycloalkyl R₁ can comprise a radical selected in a group consisting of:

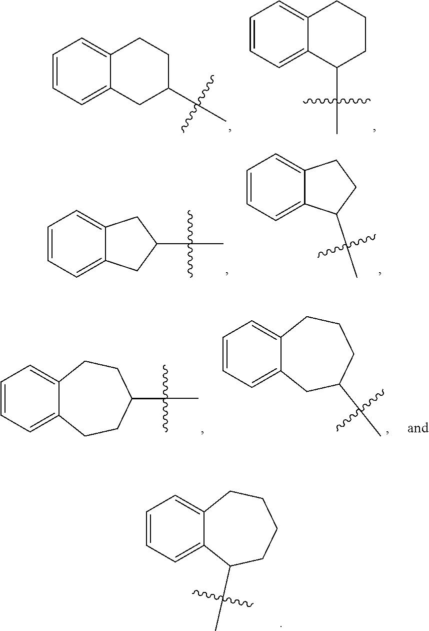

Alternatively, the optionally substituted fused arylcycloalkyl R₁ can comprise a radical selected in a group consisting of:

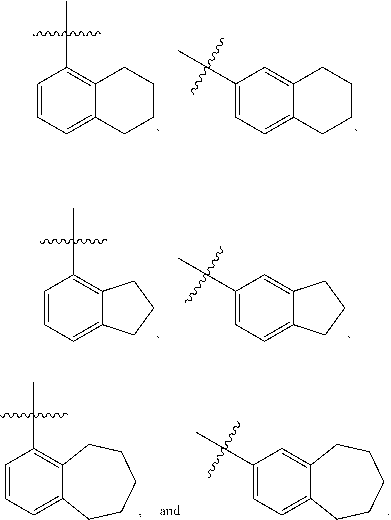 [SMILES: *c1cccc2c1CCCC2 ; *c1ccc2c(c1)CCCC2 ; *c1cccc2c1CCC2 ; *c1ccc2c(c1)CCC2 ; *c1cccc2c1CCCCC2 ; *c1ccc2c(c1)CCCCC2]

In a particular aspect, R₁ is

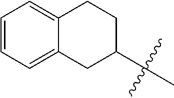 [SMILES: *C1CCc2ccccc2C1]

or a substituted radical thereof.

In a very specific aspect of the disclosure, R₁ represents

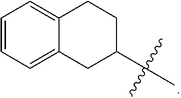 [SMILES: *C1CCc2ccccc2C1]

In a particular embodiment, as R₁ is

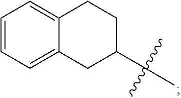 [SMILES: *C1CCc2ccccc2C1]

R₂ is a hydrogen or a halogen, preferably a halogen such as F or CI; R₁ is a phenyl optionally substituted by a halogen such as F or CI; and R₅ is a hydrogen.

In a preferred embodiment, the compound according to the present invention is selected in the group consisting of compounds of the table A below:

TABLE A

| | |
|---|---|
| 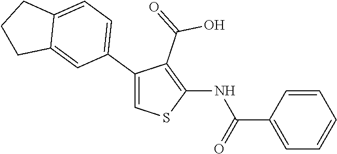 [SMILES: O=C(Nc1scc(-c2ccc3c(c2)CCC3)c1C(=O)O)c1ccccc1] | Compound #3 |
| 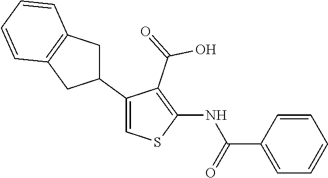 [SMILES: O=C(Nc1scc(C2Cc3ccccc3C2)c1C(=O)O)c1ccccc1] | Compound #7 |
| 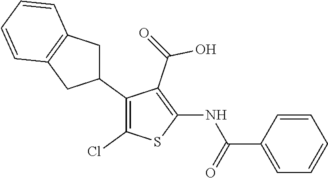 [SMILES: O=C(Nc1sc(Cl)c(C2Cc3ccccc3C2)c1C(=O)O)c1ccccc1] | Compound #8 |
| 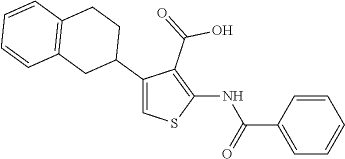 [SMILES: O=C(Nc1scc(C2CCc3ccccc3C2)c1C(=O)O)c1ccccc1] | Compound #16 |
| 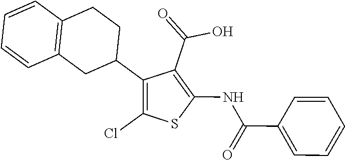 [SMILES: O=C(Nc1sc(Cl)c(C2CCc3ccccc3C2)c1C(=O)O)c1ccccc1] | Compound #17 |
| 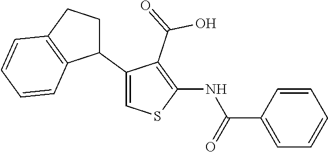 [SMILES: O=C(Nc1scc(C2CCc3ccccc32)c1C(=O)O)c1ccccc1] | Compound #19 |
| 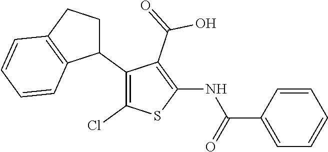 [SMILES: O=C(Nc1sc(Cl)c(C2CCc3ccccc32)c1C(=O)O)c1ccccc1] | Compound #49 |
| 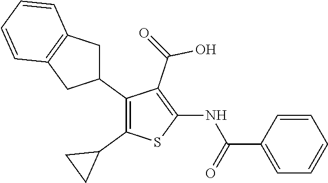 [SMILES: O=C(Nc1sc(C2CC2)c(C2Cc3ccccc3C2)c1C(=O)O)c1ccccc1] | Compound #51 |
| 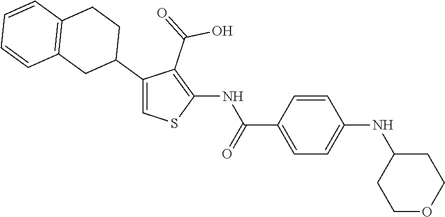 [SMILES: O=C(Nc1scc(C2CCc3ccccc3C2)c1C(=O)O)c1ccc(NC2CCOCC2)cc1] | Compound #52 |
| 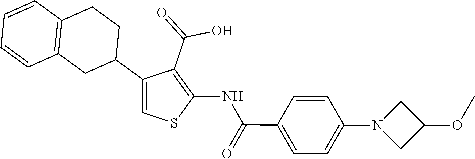 [SMILES: COC1CN(c2ccc(C(=O)Nc3scc(C4CCc5ccccc5C4)c3C(=O)O)cc2)C1] | Compound #53 |
| 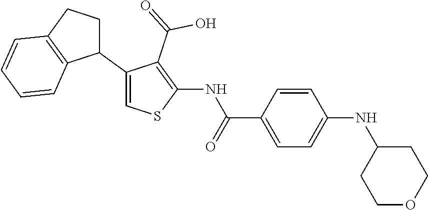 [SMILES: O=C(Nc1scc(C2CCc3ccccc32)c1C(=O)O)c1ccc(NC2CCOCC2)cc1] | Compound #55 |
| 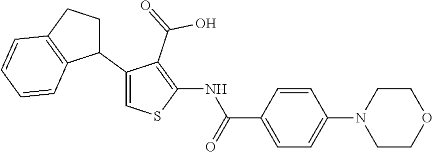 [SMILES: O=C(Nc1scc(C2CCc3ccccc32)c1C(=O)O)c1ccc(N2CCOCC2)cc1] | Compound #56 |
| 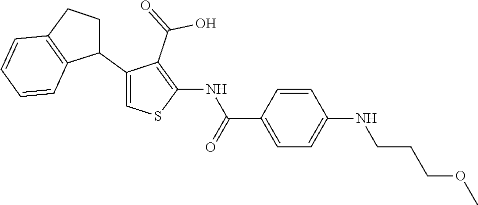 [SMILES: COCCCNc1ccc(C(=O)Nc2scc(C3CCc4ccccc43)c2C(=O)O)cc1] | Compound #57 |
| 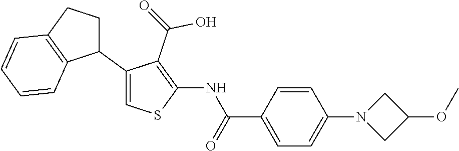 [SMILES: COC1CN(c2ccc(C(=O)Nc3scc(C4CCc5ccccc54)c3C(=O)O)cc2)C1] | Compound #58 |
| 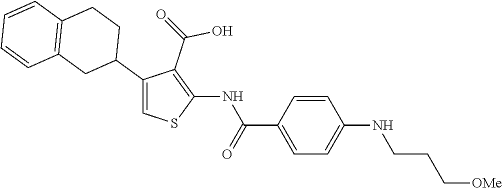 [SMILES: COCCCNc1ccc(C(=O)Nc2scc(C3CCc4ccccc4C3)c2C(=O)O)cc1] | Compound #64 |
| 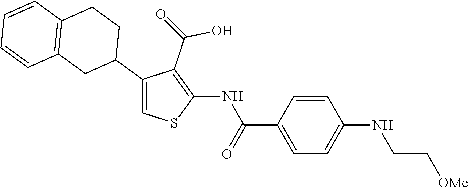 [SMILES: COCCNc1ccc(C(=O)Nc2scc(C3CCc4ccccc4C3)c2C(=O)O)cc1] | Compound #65 |
| 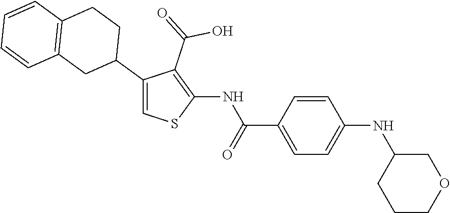 [SMILES: O=C(Nc1scc(C2CCc3ccccc3C2)c1C(=O)O)c1ccc(NC2CCCOC2)cc1] | Compound #67 |
| 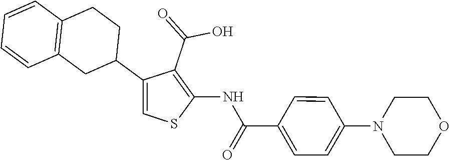 [SMILES: O=C(Nc1scc(C2CCc3ccccc3C2)c1C(=O)O)c1ccc(N2CCOCC2)cc1] | Compound #68 |
| 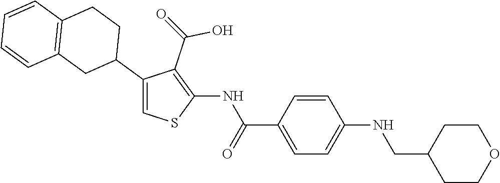 [SMILES: O=C(Nc1scc(C2CCc3ccccc3C2)c1C(=O)O)c1ccc(NCC2CCOCC2)cc1] | Compound #69 |
| 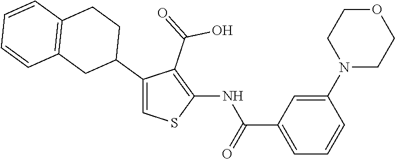 [SMILES: O=C(Nc1scc(C2CCc3ccccc3C2)c1C(=O)O)c1cccc(N2CCOCC2)c1] | Compound #70 |
| 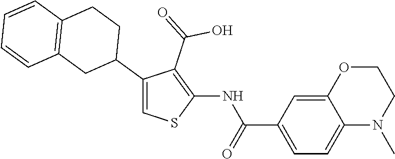 [SMILES: CN1CCOc2cc(C(=O)Nc3scc(C4CCc5ccccc5C4)c3C(=O)O)ccc21] | Compound #86 |
| 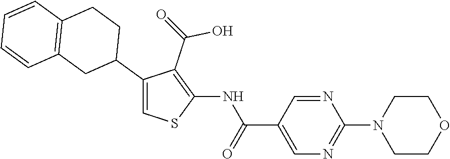 [SMILES: O=C(Nc1scc(C2CCc3ccccc3C2)c1C(=O)O)c1cnc(N2CCOCC2)nc1] | Compound #89 |
| 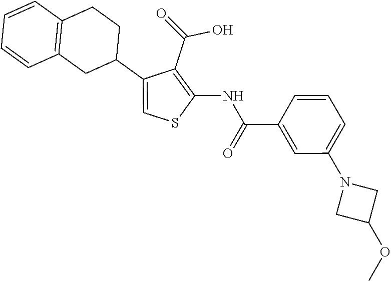 [SMILES: COC1CN(c2cccc(C(=O)Nc3scc(C4CCc5ccccc5C4)c3C(=O)O)c2)C1] | Compound #93 |
| 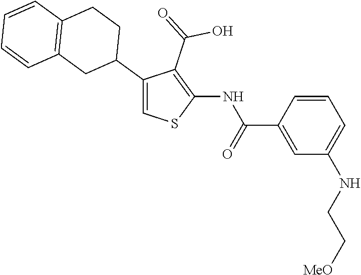 [SMILES: COCCNc1cccc(C(=O)Nc2scc(C3CCc4ccccc4C3)c2C(=O)O)c1] | Compound #94 |
| 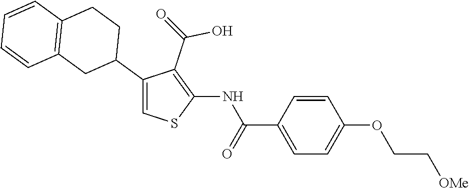 [SMILES: COCCOc1ccc(C(=O)Nc2scc(C3CCc4ccccc4C3)c2C(=O)O)cc1] | Compound #101 |
| 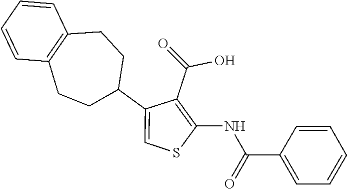 [SMILES: O=C(Nc1scc(C2CCc3ccccc3CC2)c1C(=O)O)c1ccccc1] | Compound #122 |
| 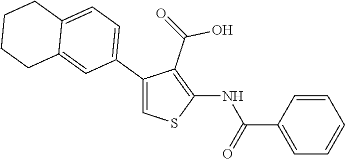 [SMILES: O=C(Nc1scc(-c2ccc3c(c2)CCCC3)c1C(=O)O)c1ccccc1] | Compound #123 |
| 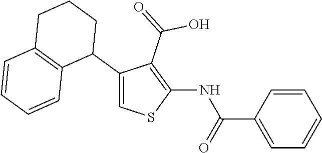 [SMILES: O=C(Nc1scc(C2CCCc3ccccc32)c1C(=O)O)c1ccccc1] | Compound #124 |
| 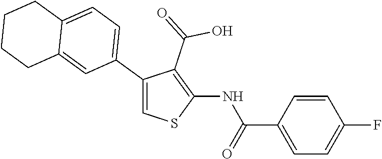 [SMILES: O=C(Nc1scc(-c2ccc3c(c2)CCCC3)c1C(=O)O)c1ccc(F)cc1] | Compound #125 |
| 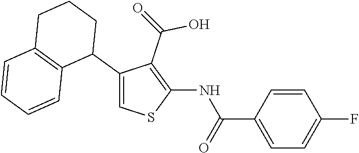 [SMILES: O=C(Nc1scc(C2CCCc3ccccc32)c1C(=O)O)c1ccc(F)cc1] | Compound #126 |
| 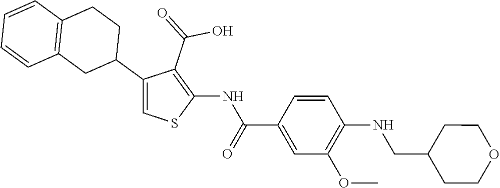 [SMILES: COc1cc(C(=O)Nc2scc(C3CCc4ccccc4C3)c2C(=O)O)ccc1NCC1CCOCC1] | Compound #127 |
| 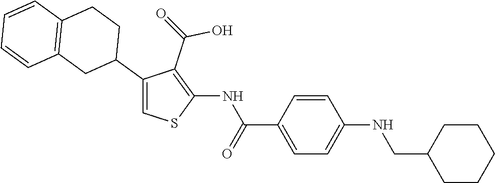 [SMILES: O=C(Nc1scc(C2CCc3ccccc3C2)c1C(=O)O)c1ccc(NCC2CCCCC2)cc1] | Compound #128 |
| 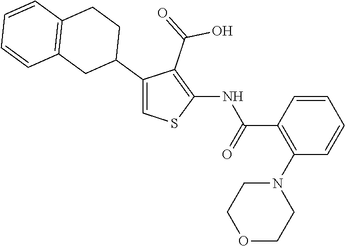 [SMILES: O=C(Nc1scc(C2CCc3ccccc3C2)c1C(=O)O)c1ccccc1N1CCOCC1] | Compound #129 |
| 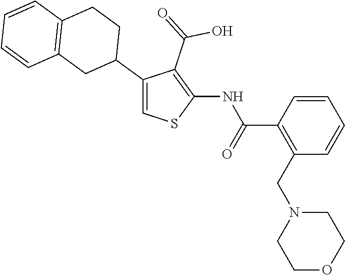 [SMILES: O=C(Nc1scc(C2CCc3ccccc3C2)c1C(=O)O)c1ccccc1CN1CCOCC1] | Compound #130 |
| 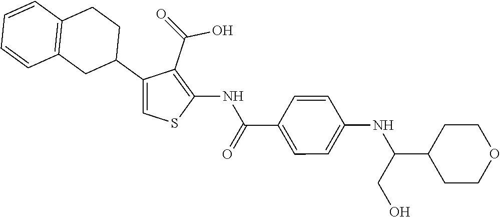 [SMILES: O=C(Nc1scc(C2CCc3ccccc3C2)c1C(=O)O)c1ccc(NC(CO)C2CCOCC2)cc1] | Compound #131 |
| 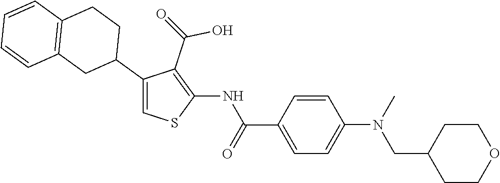 [SMILES: CN(CC1CCOCC1)c1ccc(C(=O)Nc2scc(C3CCc4ccccc4C3)c2C(=O)O)cc1] | Compound #132 |
| 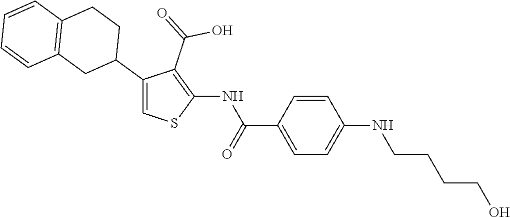 [SMILES: O=C(Nc1scc(C2CCc3ccccc3C2)c1C(=O)O)c1ccc(NCCCCO)cc1] | Compound #133 |
| 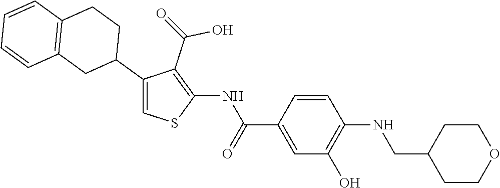 [SMILES: O=C(Nc1scc(C2CCc3ccccc3C2)c1C(=O)O)c1ccc(NCC2CCOCC2)c(O)c1] | Compound #134 |
| 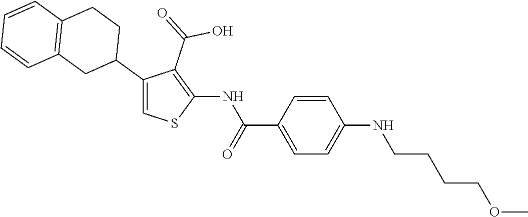 | Compound #135 |
| 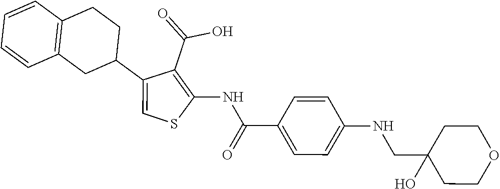 [SMILES: O=C(Nc1scc(C2CCc3ccccc3C2)c1C(=O)O)c1ccc(NCC2(O)CCOCC2)cc1] | Compound #136 |
| 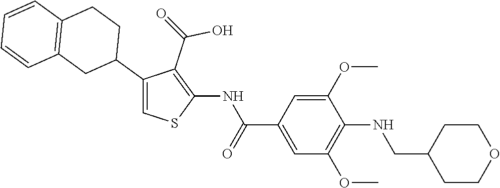 [SMILES: COc1cc(C(=O)Nc2scc(C3CCc4ccccc4C3)c2C(=O)O)cc(OC)c1NCC1CCOCC1] | Compound #137 |
| 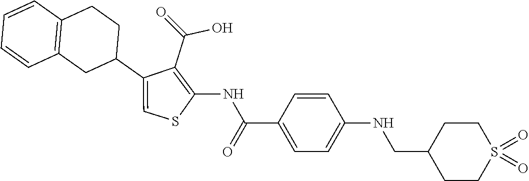 [SMILES: O=C(Nc1scc(C2CCc3ccccc3C2)c1C(=O)O)c1ccc(NCC2CCS(=O)(=O)CC2)cc1] | Compound #138 |
| 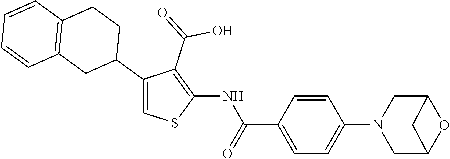 [SMILES: O=C(Nc1scc(C2CCc3ccccc3C2)c1C(=O)O)c1ccc(N2CC3CC(C2)O3)cc1] | Compound #139 |
| 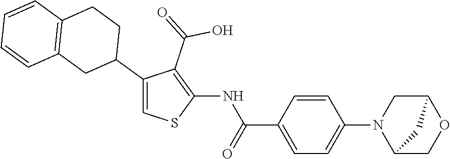 [SMILES: O=C(Nc1scc(C2CCc3ccccc3C2)c1C(=O)O)c1ccc(N2C[C@@H]3C[C@H]2CO3)cc1] | Compound #140 |
| 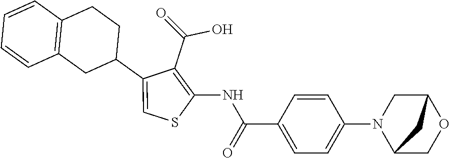 [SMILES: O=C(Nc1scc(C2CCc3ccccc3C2)c1C(=O)O)c1ccc(N2C[C@H]3C[C@@H]2CO3)cc1] | Compound #141 |
| 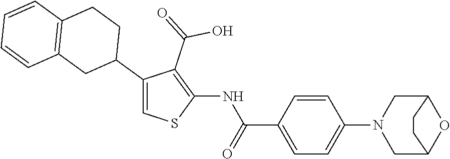 [SMILES: O=C(Nc1scc(C2CCc3ccccc3C2)c1C(=O)O)c1ccc(N2CC3CCC(C2)O3)cc1] | Compound #142 |
| 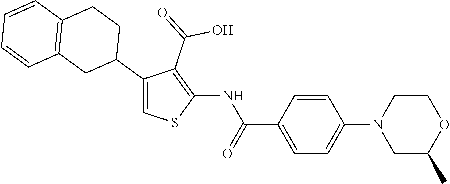 [SMILES: C[C@H]1CN(c2ccc(C(=O)Nc3scc(C4CCc5ccccc5C4)c3C(=O)O)cc2)CCO1] | Compound #143 |
| 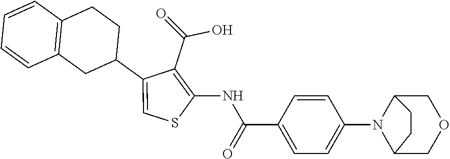 [SMILES: O=C(Nc1scc(C2CCc3ccccc3C2)c1C(=O)O)c1ccc(N2C3CCC2COC3)cc1] | Compound #144 |
| 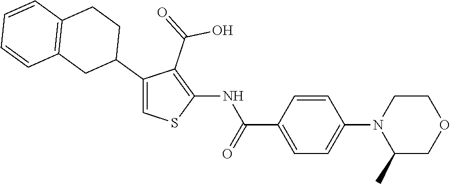 [SMILES: C[C@@H]1COCCN1c1ccc(C(=O)Nc2scc(C3CCc4ccccc4C3)c2C(=O)O)cc1] | Compound #145 |
| 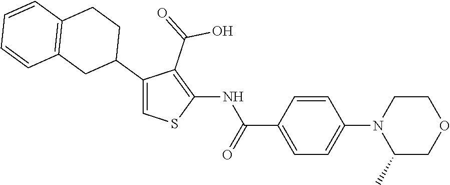 [SMILES: C[C@H]1COCCN1c1ccc(C(=O)Nc2scc(C3CCc4ccccc4C3)c2C(=O)O)cc1] | Compound #146 |
| 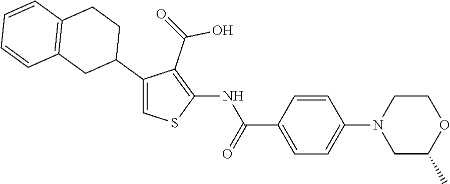 [SMILES: C[C@@H]1CN(c2ccc(C(=O)Nc3scc(C4CCc5ccccc5C4)c3C(=O)O)cc2)CCO1] | Compound #147 |
| 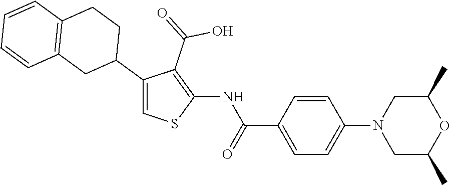 [SMILES: C[C@@H]1CN(c2ccc(C(=O)Nc3scc(C4CCc5ccccc5C4)c3C(=O)O)cc2)C[C@H](C)O1] | Compound #148 |
| 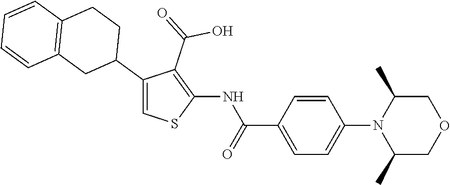 [SMILES: C[C@@H]1COC[C@H](C)N1c1ccc(C(=O)Nc2scc(C3CCc4ccccc4C3)c2C(=O)O)cc1] | Compound #149 |
| 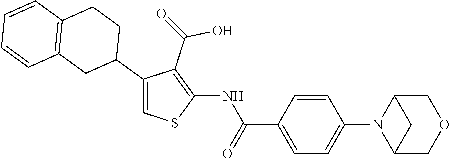 [SMILES: O=C(Nc1scc(C2CCc3ccccc3C2)c1C(=O)O)c1ccc(N2C3COCC2C3)cc1] | Compound #150 |
| 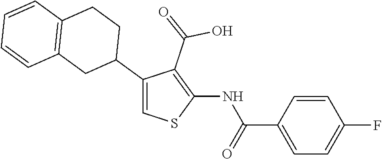 [SMILES: O=C(Nc1scc(C2CCc3ccccc3C2)c1C(=O)O)c1ccc(F)cc1] | Compound #151 |
| 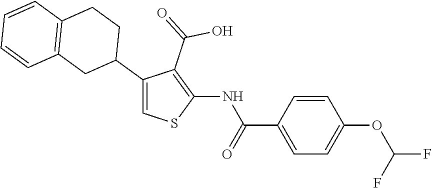 [SMILES: O=C(Nc1scc(C2CCc3ccccc3C2)c1C(=O)O)c1ccc(OC(F)F)cc1] | Compound #152 |
| 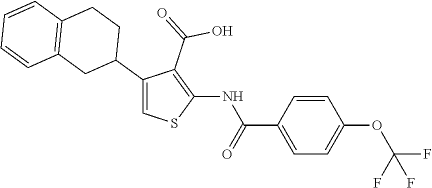 [SMILES: O=C(Nc1scc(C2CCc3ccccc3C2)c1C(=O)O)c1ccc(OC(F)(F)F)cc1] | Compound #153 |
| 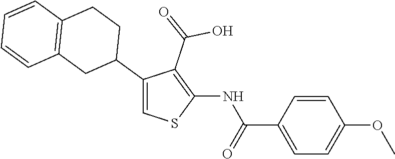 [SMILES: COc1ccc(C(=O)Nc2scc(C3CCc4ccccc4C3)c2C(=O)O)cc1] | Compound #154 |
| 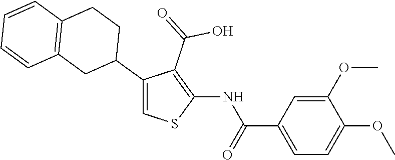 [SMILES: COc1ccc(C(=O)Nc2scc(C3CCc4ccccc4C3)c2C(=O)O)cc1OC] | Compound #155 |
| 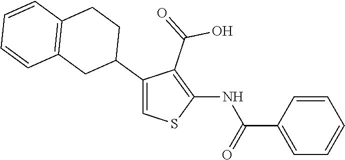 [SMILES: O=C(Nc1scc(C2CCc3ccccc3C2)c1C(=O)O)c1ccccc1] | Compound #156 |
| 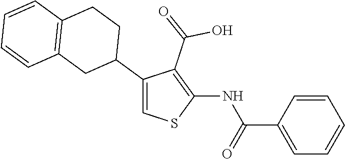 [SMILES: O=C(Nc1scc(C2CCc3ccccc3C2)c1C(=O)O)c1ccccc1] | Compound #157 |
| 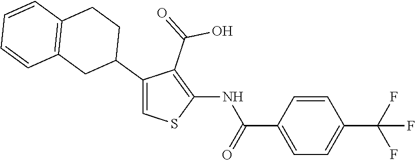 [SMILES: O=C(Nc1scc(C2CCc3ccccc3C2)c1C(=O)O)c1ccc(C(F)(F)F)cc1] | Compound #158 |
| 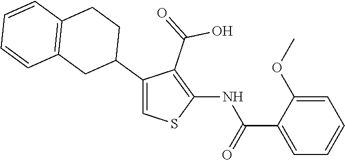 [SMILES: COc1ccccc1C(=O)Nc1scc(C2CCc3ccccc3C2)c1C(=O)O] | Compound #159 |
| 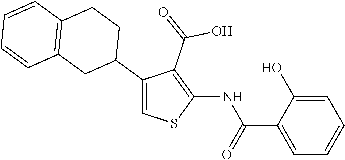 [SMILES: O=C(Nc1scc(C2CCc3ccccc3C2)c1C(=O)O)c1ccccc1O] | Compound #160 |
| 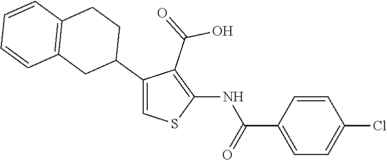 [SMILES: O=C(Nc1scc(C2CCc3ccccc3C2)c1C(=O)O)c1ccc(Cl)cc1] | Compound #161 |
| 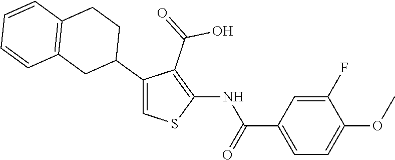 [SMILES: COc1ccc(C(=O)Nc2scc(C3CCc4ccccc4C3)c2C(=O)O)cc1F] | Compound #162 |
| 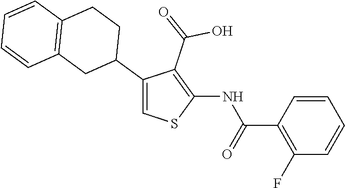 [SMILES: O=C(Nc1scc(C2CCc3ccccc3C2)c1C(=O)O)c1ccccc1F] | Compound #163 |
| 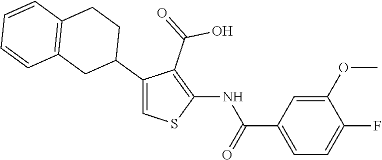 [SMILES: COc1cc(C(=O)Nc2scc(C3CCc4ccccc4C3)c2C(=O)O)ccc1F] | Compound #164 |
| 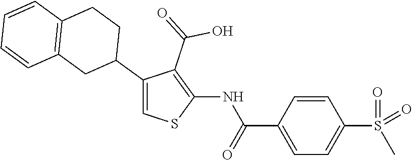 [SMILES: CS(=O)(=O)c1ccc(C(=O)Nc2scc(C3CCc4ccccc4C3)c2C(=O)O)cc1] | Compound #165 |
| 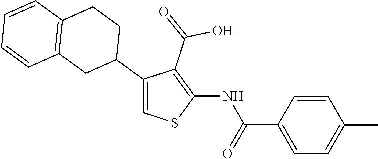 [SMILES: Cc1ccc(C(=O)Nc2scc(C3CCc4ccccc4C3)c2C(=O)O)cc1] | Compound #166 |
| 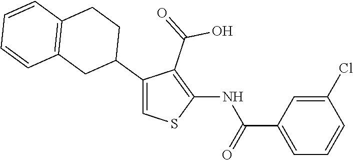 [SMILES: O=C(Nc1scc(C2CCc3ccccc3C2)c1C(=O)O)c1cccc(Cl)c1] | Compound #167 |
| 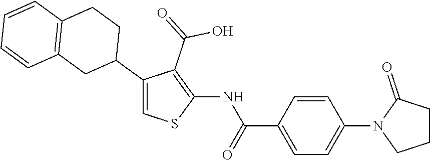 [SMILES: O=C(Nc1scc(C2CCc3ccccc3C2)c1C(=O)O)c1ccc(N2CCCC2=O)cc1] | Compound #168 |
| 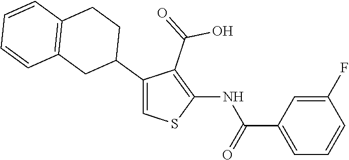 [SMILES: O=C(Nc1scc(C2CCc3ccccc3C2)c1C(=O)O)c1cccc(F)c1] | Compound #169 |
| 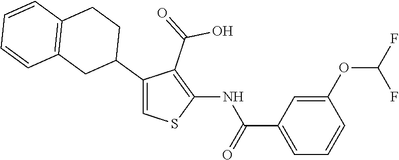 [SMILES: O=C(Nc1scc(C2CCc3ccccc3C2)c1C(=O)O)c1cccc(OC(F)F)c1] | Compound #170 |
| 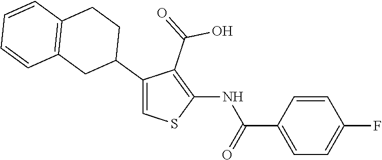 [SMILES: O=C(Nc1scc(C2CCc3ccccc3C2)c1C(=O)O)c1ccc(F)cc1] | Compound #171 |
| 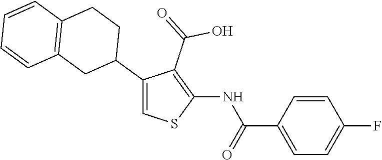 [SMILES: O=C(Nc1scc(C2CCc3ccccc3C2)c1C(=O)O)c1ccc(F)cc1] | Compound #172 |
| 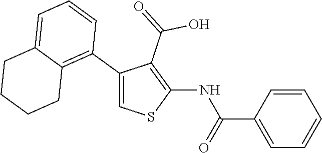 [SMILES: O=C(Nc1scc(-c2cccc3c2CCCC3)c1C(=O)O)c1ccccc1] | Compound #194 |
| 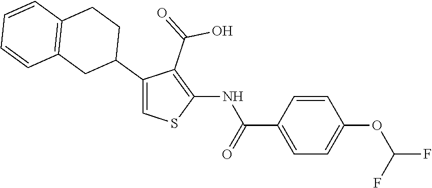 [SMILES: O=C(Nc1scc(C2CCc3ccccc3C2)c1C(=O)O)c1ccc(OC(F)F)cc1] | Compound #174 |
| 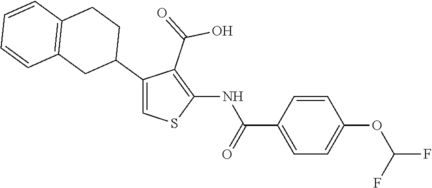 [SMILES: O=C(Nc1scc(C2CCc3ccccc3C2)c1C(=O)O)c1ccc(OC(F)F)cc1] | Compound #175 |
| 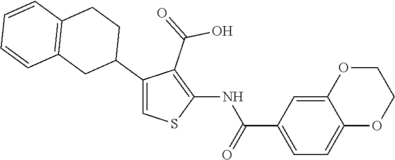 [SMILES: O=C(Nc1scc(C2CCc3ccccc3C2)c1C(=O)O)c1ccc2c(c1)OCCO2] | Compound #176 |
| 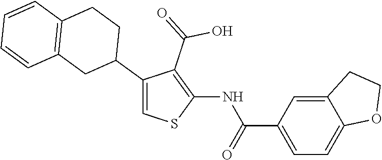 [SMILES: O=C(Nc1scc(C2CCc3ccccc3C2)c1C(=O)O)c1ccc2c(c1)CCO2] | Compound #177 |
| 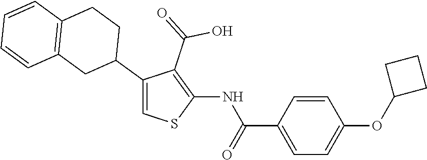 [SMILES: O=C(Nc1scc(C2CCc3ccccc3C2)c1C(=O)O)c1ccc(OC2CCC2)cc1] | Compound #178 |
| 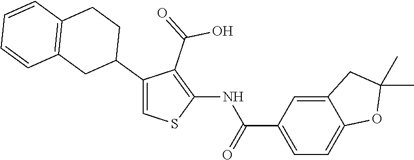 [SMILES: CC1(C)Cc2cc(C(=O)Nc3scc(C4CCc5ccccc5C4)c3C(=O)O)ccc2O1] | Compound #179 |
| 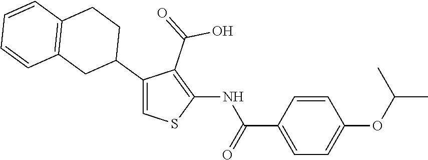 [SMILES: CC(C)Oc1ccc(C(=O)Nc2scc(C3CCc4ccccc4C3)c2C(=O)O)cc1] | Compound #180 |
| 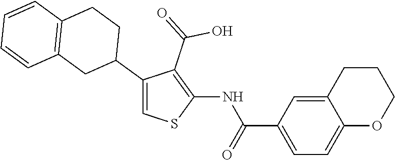 [SMILES: O=C(Nc1scc(C2CCc3ccccc3C2)c1C(=O)O)c1ccc2c(c1)CCCO2] | Compound #181 |
| 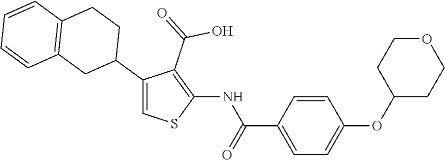 [SMILES: O=C(Nc1scc(C2CCc3ccccc3C2)c1C(=O)O)c1ccc(OC2CCOCC2)cc1] | Compound #182 |
| 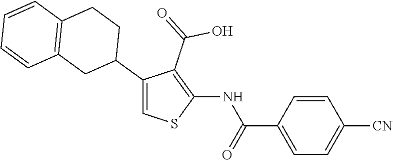 [SMILES: N#Cc1ccc(C(=O)Nc2scc(C3CCc4ccccc4C3)c2C(=O)O)cc1] | Compound #183 |
| 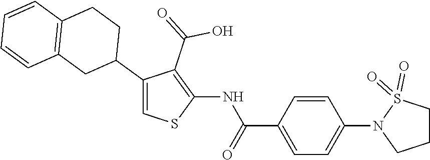 [SMILES: O=C(Nc1scc(C2CCc3ccccc3C2)c1C(=O)O)c1ccc(N2CCCS2(=O)=O)cc1] | Compound #184 |
| 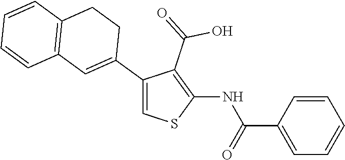 [SMILES: O=C(Nc1scc(C2=Cc3ccccc3CC2)c1C(=O)O)c1ccccc1] | Compound #185 |
| 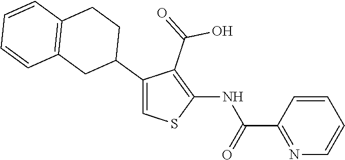 [SMILES: O=C(Nc1scc(C2CCc3ccccc3C2)c1C(=O)O)c1ccccn1] | Compound #186 |
| 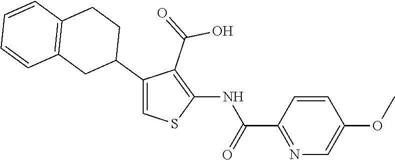 [SMILES: COc1ccc(C(=O)Nc2scc(C3CCc4ccccc4C3)c2C(=O)O)nc1] | Compound #187 |
| 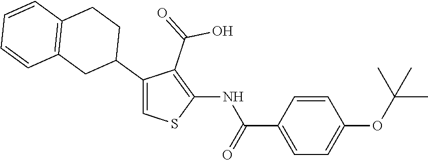 [SMILES: CC(C)(C)Oc1ccc(C(=O)Nc2scc(C3CCc4ccccc4C3)c2C(=O)O)cc1] | Compound #188 |
| 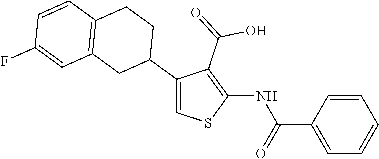 [SMILES: O=C(Nc1scc(C2CCc3ccc(F)cc3C2)c1C(=O)O)c1ccccc1] | Compound #189 |
| 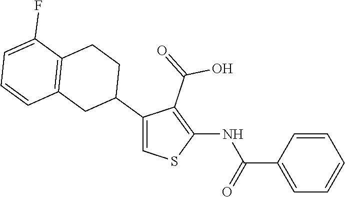 [SMILES: O=C(Nc1scc(C2CCc3c(F)cccc3C2)c1C(=O)O)c1ccccc1] | Compound #190 |
| 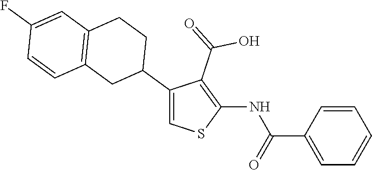 [SMILES: O=C(Nc1scc(C2CCc3cc(F)ccc3C2)c1C(=O)O)c1ccccc1] | Compound #191 |
| 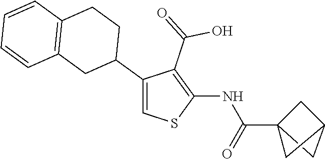 [SMILES: O=C(O)c1c(C2CCc3ccccc3C2)csc1NC(=O)C12CC(C1)C2] | Compound #192 |
| 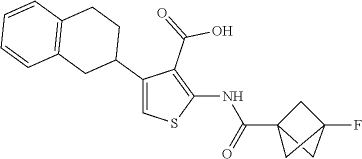 [SMILES: O=C(O)c1c(C2CCc3ccccc3C2)csc1NC(=O)C12CC(F)(C1)C2] | Compound #193 |
| 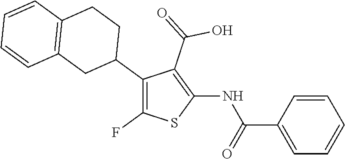 [SMILES: O=C(Nc1sc(F)c(C2CCc3ccccc3C2)c1C(=O)O)c1ccccc1] | Compound #194 |
| 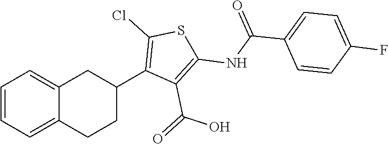 [SMILES: O=C(Nc1sc(Cl)c(C2CCc3ccccc3C2)c1C(=O)O)c1ccc(F)cc1] | Compound #195 |
| 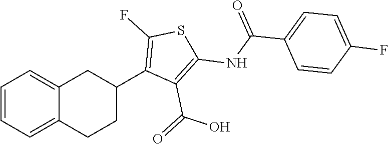 [SMILES: O=C(Nc1sc(F)c(C2CCc3ccccc3C2)c1C(=O)O)c1ccc(F)cc1] | Compound #196 |

In a very particular aspect, the compound is selected from the group consisting of compounds #16, #17, #151, #157, #171, #194, #195 and #196.

In the second aspect, R₁ represents an optionally substituted cycloalkyl.

In a particular aspect, R₁ is a radical selected in the group consisting of:

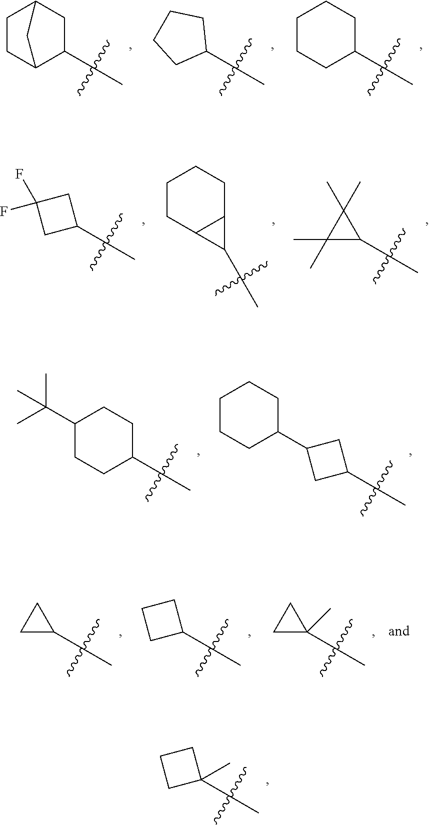

In a preferred aspect, the compound is selected in the group consisting of compounds of the table B below:

TABLE B

| | |
|---|---|
| 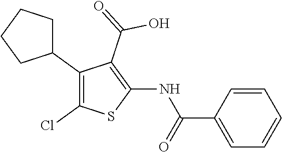 [SMILES: O=C(Nc1sc(Cl)c(C2CCCC2)c1C(=O)O)c1ccccc1] | Compound #B18 |
| 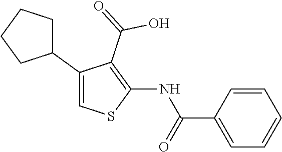 [SMILES: O=C(Nc1scc(C2CCCC2)c1C(=O)O)c1ccccc1] | Compound #B10 |
| 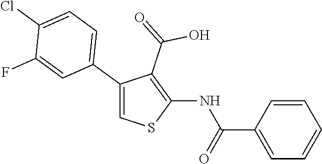 [SMILES: O=C(Nc1scc(-c2ccc(Cl)c(F)c2)c1C(=O)O)c1ccccc1] | Compound #B2 |
| 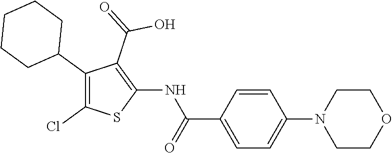 [SMILES: O=C(Nc1sc(Cl)c(C2CCCCC2)c1C(=O)O)c1ccc(N2CCOCC2)cc1] | Compound #B15 |
| 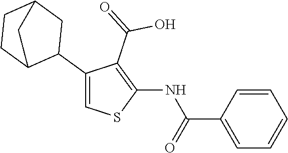 [SMILES: O=C(Nc1scc(C2CC3CCC2C3)c1C(=O)O)c1ccccc1] | Compound #B9 |
| 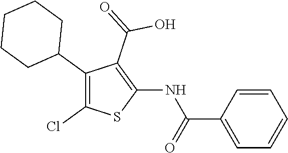 [SMILES: O=C(Nc1sc(Cl)c(C2CCCCC2)c1C(=O)O)c1ccccc1] | Compound #B12 |
| 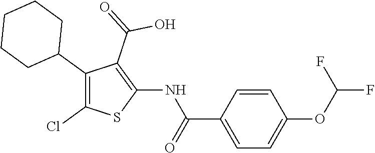 [SMILES: O=C(Nc1sc(Cl)c(C2CCCCC2)c1C(=O)O)c1ccc(OC(F)F)cc1] | Compound #B13 |
| 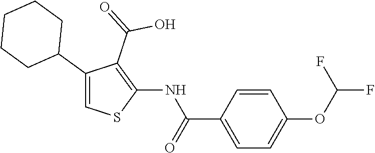 [SMILES: O=C(Nc1scc(C2CCCCC2)c1C(=O)O)c1ccc(OC(F)F)cc1] | Compound #B11 |
| 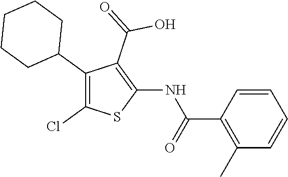 [SMILES: Cc1ccccc1C(=O)Nc1sc(Cl)c(C2CCCCC2)c1C(=O)O] | Compound #B14 |
| 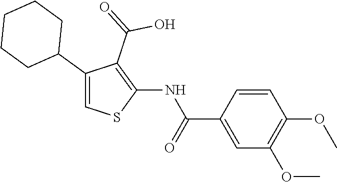 [SMILES: COc1ccc(C(=O)Nc2scc(C3CCCCC3)c2C(=O)O)cc1OC] | Compound #B25 |
| 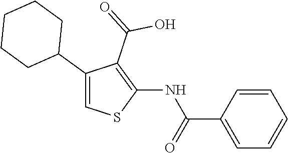 [SMILES: O=C(Nc1scc(C2CCCCC2)c1C(=O)O)c1ccccc1] | Compound #B26 |
| 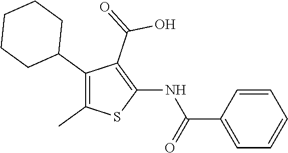 [SMILES: Cc1sc(NC(=O)c2ccccc2)c(C(=O)O)c1C1CCCCC1] | Compound #B27 |
| 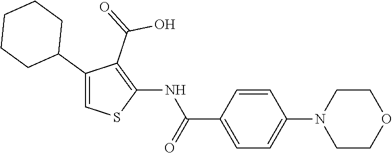 [SMILES: O=C(Nc1scc(C2CCCCC2)c1C(=O)O)c1ccc(N2CCOCC2)cc1] | Compound #B29 |
| 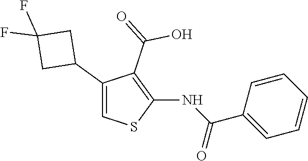 [SMILES: O=C(Nc1scc(C2CC(F)(F)C2)c1C(=O)O)c1ccccc1] | Compound #B35 |
| 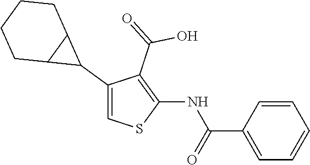 [SMILES: O=C(Nc1scc(C2C3CCCCC32)c1C(=O)O)c1ccccc1] | Compound #B37 |
| 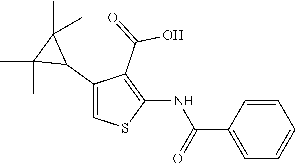 [SMILES: CC1(C)C(c2csc(NC(=O)c3ccccc3)c2C(=O)O)C1(C)C] | Compound #B39 |
| 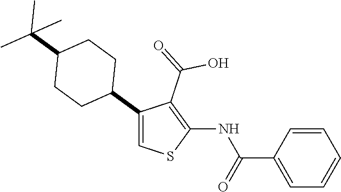 | Compound #B54 |
| 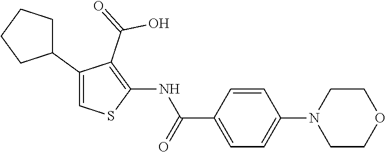 [SMILES: O=C(Nc1scc(C2CCCC2)c1C(=O)O)c1ccc(N2CCOCC2)cc1] | Compound #B59 |
| 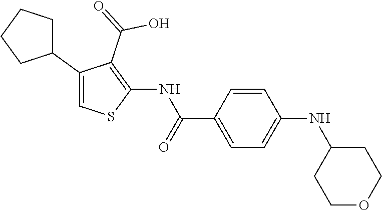 [SMILES: O=C(Nc1scc(C2CCCC2)c1C(=O)O)c1ccc(NC2CCOCC2)cc1] | Compound #B60 |
| 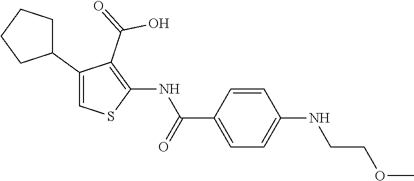 [SMILES: COCCNc1ccc(C(=O)Nc2scc(C3CCCC3)c2C(=O)O)cc1] | Compound #B61 |
| 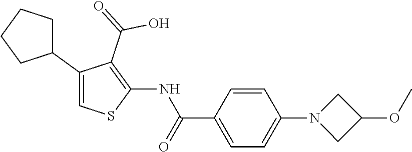 [SMILES: COC1CN(c2ccc(C(=O)Nc3scc(C4CCCC4)c3C(=O)O)cc2)C1] | Compound #B62 |
| 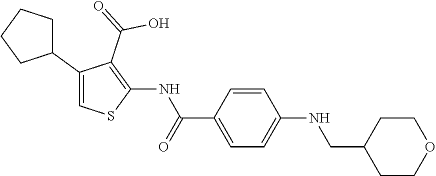 [SMILES: O=C(Nc1scc(C2CCCC2)c1C(=O)O)c1ccc(NCC2CCOCC2)cc1] | Compound #B66 |
| 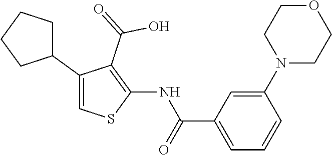 [SMILES: O=C(Nc1scc(C2CCCC2)c1C(=O)O)c1cccc(N2CCOCC2)c1] | Compound #B71 |
| 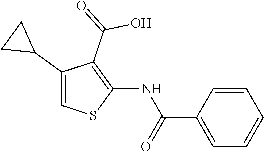 [SMILES: O=C(Nc1scc(C2CC2)c1C(=O)O)c1ccccc1] | Compound #B87 |
| 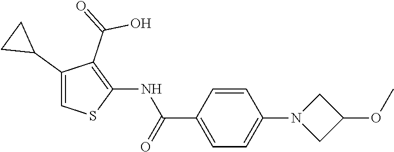 [SMILES: COC1CN(c2ccc(C(=O)Nc3scc(C4CC4)c3C(=O)O)cc2)C1] | Compound #B88 |
| 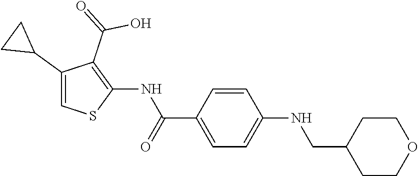 [SMILES: O=C(Nc1scc(C2CC2)c1C(=O)O)c1ccc(NCC2CCOCC2)cc1] | Compound #B90 |
| 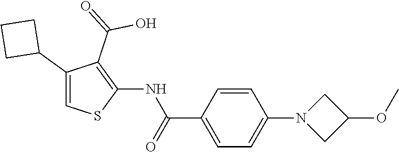 [SMILES: COC1CN(c2ccc(C(=O)Nc3scc(C4CCC4)c3C(=O)O)cc2)C1] | Compound #B97 |
| 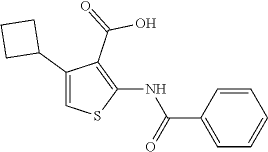 [SMILES: O=C(Nc1scc(C2CCC2)c1C(=O)O)c1ccccc1] | Compound #B98 |
| 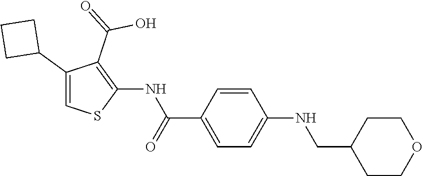 [SMILES: O=C(Nc1scc(C2CCC2)c1C(=O)O)c1ccc(NCC2CCOCC2)cc1] | Compound #B99 |
| 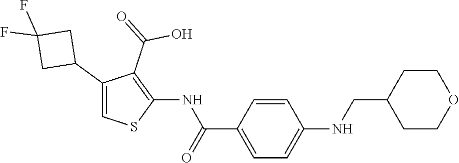 [SMILES: O=C(Nc1scc(C2CC(F)(F)C2)c1C(=O)O)c1ccc(NCC2CCOCC2)cc1] | Compound #B106 |
| 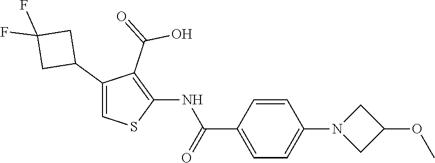 [SMILES: COC1CN(c2ccc(C(=O)Nc3scc(C4CC(F)(F)C4)c3C(=O)O)cc2)C1] | Compound #B107 |
| 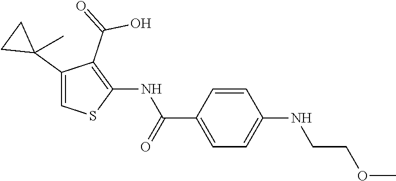 [SMILES: COCCNc1ccc(C(=O)Nc2scc(C3(C)CC3)c2C(=O)O)cc1] | Compound #B108 |
| 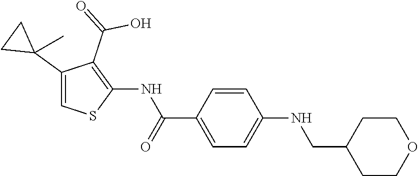 [SMILES: CC1(c2csc(NC(=O)c3ccc(NCC4CCOCC4)cc3)c2C(=O)O)CC1] | Compound #B111 |
| 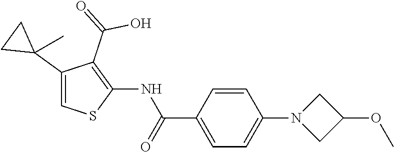 [SMILES: COC1CN(c2ccc(C(=O)Nc3scc(C4(C)CC4)c3C(=O)O)cc2)C1] | Compound #B112 |
| 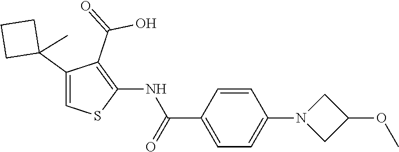 [SMILES: COC1CN(c2ccc(C(=O)Nc3scc(C4(C)CCC4)c3C(=O)O)cc2)C1] | Compound #B115 |

The present invention relates to the use of any one of these compounds.

### Diseases

The compounds of the present invention show a specific effect on hepatic periportal/portal inflammation. Optionally, they further present an effect on hepatic periportal fibrosis.

In a particular aspect, the compounds of the present invention are able to decrease the periportal/portal inflammation. For example, the decrease could be of 20, 30, 40, 50, 60, 70, 80, 90 or 100% in comparison of the portal/periportal inflammation in absence of treatment with the compound. The decrease can be measured by any method available for the person skilled in the art, for instance in an animal model as detailed in the example.

In an additional aspect, the compounds of the present invention are able to decrease the portal/periportal fibrosis. For example, the decrease could be of 20, 30, 40, 50, 60, 70, 80, 90 or 100% in comparison of the portal/periportal fibrosis in absence of treatment with the compound. The decrease can be measured in an animal model as detailed in the example.

Hepatic portal inflammation is involved in several diseases.

Accordingly, the disease can be selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury or chronic drug toxicity, biliary atresia, idiopathic neonatal hepatitis syndrome, progressive familial intrahepatic cholestasis (PFIC) and primary and secondary mitochondrial hepatopathies.

Non-alcoholic fatty liver disease (NAFLD) is the most common cause of chronic liver disease in children. NAFLD includes a range of disease states from benign steatosis to non-alcoholic steatohepatitis (NASH). The disease often leads to cirrhosis of the liver resulting in the need for liver transplantation as well as causing other problems including, but not limited to, metabolic and cardiovascular disease. Although the pathogenesis of NAFLD is still unclear, it is likely that insulin resistance, increased oxidative stress and lipid peroxidation play roles. Levels of intracellular glutathione, which protects against oxidative stress, are low in NAFLD subjects. 18 years old, in particular between 8 years old and 17 years old.

Two distinct histological forms of NASH have been described. Type 1 NASH occurs in adults and some children and is characterized by steatosis, lobular inflammation, ballooning degeneration and perisinusoidal fibrosis. Type 2 NASH, found most commonly in children and juveniles, is characterized by steatosis, portal inflammation, and portal fibrosis. Schwimmer et al. (Hepatgology, 42 (3): 641-649, 2005; incorporated herein by reference) described various criteria and biomarkers used to differentiate NASH Type 1 from NASH Type 2. As stated above, Type 1 NASH demonstrates a prevalent lobular inflammation in the liver in contrast with a prevalent portal inflammation in Type 2 NASH. NAFLD and NASH can be scored by use of criteria outlined by Kleiner et al. (Hepatology 41, p.1313-21, 2005).

Therefore, in a particular aspect, the NASH is a pediatric NASH. Then, the subject to be treated is less than 18 years old, in particular between 8 years old and 17 years old.

Primary and secondary mitochondrial hepatopathies are conditions divided into primary (caused by specific gene mutations) and secondary (where mitochondria are targeted by endogenous or exogenous toxins) (Bandyopadhyay and Dutta, 2005, JAPI, 53, 973-978).

Autoimmune hepatitis (AIH), also called lupoid hepatitis, is a chronic, autoimmune disease of the liver. Four subtypes of autoimmune hepatitis are recognized: Type 1 AlH; Type 2 AlH; Type 3 AlH and AlH with no autoantibodies detected.

Primary mitochondrial hepatopathies can be due to Electron transport (respiratory chain) defects, Fatty acid oxidation and transport defects, Carnitine palmitoyltransferase I and II deficiency, Carnitine acylcarnitine translocase deficiency, Urea cycle enzyme deficiencies, Electron transfer flavoprotein (ETF) and ETFdehydrogenase deficiencies, Phosphoenolpyruvate carboxykinase deficiency, or Non-ketotic hyperglycinemia.

Secondary mitochondrial hepatopathies include Reye's syndrome, Hepatic copper overload such as Wilson's disease, Indian childhood cirrhosis and Idiopathic infantile copper toxicosis, Neonatal iron storage disease, Type I Tyrosinemia, Zellweger syndrome, secondary mitochondrial hepatopathies due to drugs such as valproic acid, salicylic acid, nucleoside analogs, chloramphenicol and barbiturates, chemical toxins such as iron, cyanide, antimycin A and rotenone, and bacterial toxins such as Cereulide or Ekiri.

Chronic Chemical and Drug Induced Liver Injury or chronic drug toxicity is a liver disease lasting six months or more, caused by an adverse effect of a drug or chemical (see NCBI, MeSH databasis, www.ncbi.nlm.nih.gov/mesh/68056487). The adverse effect may be caused by drugs, drug metabolites, chemicals from the environment, or an idiosyncratic response.

In addition, some diseases may present a subgroup of patients having a periportal or portal inflammation. More particular, the subject has a periportal or portal inflammation of grade 1 or grade 2, as further defined below. Said diseases can be selected from the group consisting of viral hepatitis, in particular chronic viral hepatitis, hemochromatosis and NASH. Accordingly, the subject may have a periportal or portal inflammation of grade 1 or grade 2, as further defined below and suffer from a disease selected from the group consisting of viral hepatitis, in particular chronic viral hepatitis, hemochromatosis and NASH. Optionally, the subject may further have a hepatic periportal fibrosis, in particular at stage 1C, stage 2 or stage 3 as defined below.

For instance, as discussed in the background section, patients with NASH and having a periportal or portal inflammation are a subgroup of patients associated with a more severe disease. Indeed, not all patients suffering from NASH have a periportal or portal inflammation. Especially, periportal/portal inflammation is inversely correlated with survival free of liver transplantation and survival free of liver-related events. Accordingly, there is an interest to decrease periportal/portal inflammation in these patients.

Periportal/portal inflammation and fibrosis can be assessed in a hepatic biopsy.

Portal inflammation can be more specifically defined by a grade ranging from 0 to 2 as outlined by Brunt et al (Hepatology; 2009, 49, 809-820; Hepatology, 2011, 53, 810-820), the disclosure thereof being incorporated herein by reference. The grade 0 corresponds to “none”. The grade 1 corresponds to “mild”. The grade 2 corresponds to “more than mild”. In a specific aspect, the subject or patient has a mild portal inflammation (i.e., grade 1) or more than mild portal inflammation (i.e., grade 2).

Then, the subject to be treated suffers from a disease with a hepatic periportal/portal inflammation. In a particular aspect, the subject has a mild portal inflammation (i.e., grade 1) or more than mild portal inflammation (i.e., grade 2).

Optionally, the subject may further suffer from a disease with a hepatic periportal fibrosis. In a particular aspect, the subject has a portal fibrosis, e.g. a fibrosis at stage 1C (portal/periportal), stage 2 (perisinusoridal and portal/periportal) or stage 3 (bridging fibrosis). Fibrosis can be scored by use of criteria outlined by Kleiner et al. (Hepatology 41, p.1313-21, 2005, more particularly Table 1) and Brunt et al (Am J Gastroenterol 1999;94:2467-2474).

In a particular aspect, the subject is a human. In a specific aspect, the subject is a child or a juvenile. Accordingly, the subject is less than 20, 19 or 18 years old. For instance, the subject is between 10 and 20 years old, between 11 and 19 years old or between 12 and 18 years old. In another aspect, the subject is an adult.

The subject can be obese or overweight. More specifically, a subject with a BMI of 30 or more is generally considered obese and a subject with a BMI equal to or more than 25 and less than 30 is considered overweight.

In a very specific aspect, the disease is non-alcoholic fatty liver disease (NAFLD) or pediatric non-alcoholic steatohepatitis (NASH). The subject is a child or a juvenile, especially as defined above, and the subject is overweight or obese. The subject may have a high hepatic test and/or abnormal hepatic echography.

### Combinations

The compounds of the present disclosure can be used in combination with other therapeutic agents. The additional therapeutic agents can be selected from the agents already used for the treatment of one of the diseases as specified above.

In a particular aspect, the other therapeutic agents have a weak effect or no effect on portal/periportal inflammation or portal/periportal fibrosis.

In a very specific aspect, the other therapeutic agent is Obeticholic acid (OCA). Accordingly, the present invention further relates to
a pharmaceutical composition comprising a compound as disclosed herein and Obeticholic acid (OCA), in particular for use for treating a disease as specified above;
the use of a pharmaceutical composition comprising a compound as disclosed herein and Obeticholic acid (OCA), for the manufacture of a medicine for treating a disease as specified above;
a compound as disclosed herein or a pharmaceutical composition comprising a compound as disclosed herein for use for treating a disease as specified above in combination with Obeticholic acid (OCA);
the use of a compound as disclosed herein or a pharmaceutical composition comprising a compound as disclosed herein for the manufacture of a medicine for treating a disease as specified above, to be used in combination with Obeticholic acid (OCA);
a method for treating a disease as specified above in a subject, comprising administering to the subject a therapeutic amount of a pharmaceutical composition comprising a compound as disclosed herein and Obeticholic acid (OCA) or administering to the subject a therapeutic amount of a compound as disclosed herein and a therapeutic amount of Obeticholic acid (OCA).

### Pharmaceutical Composition

The pharmaceutical composition comprises a compound of the present invention and optionally at least one pharmaceutically acceptable carrier or excipient.

The compound according to the present disclosure or the pharmaceutical composition according to the present disclosure may be administered by any conventional route of administration. In particular, the compound or the pharmaceutical composition of the present disclosure can be administered by a topical, enteral, oral, parenteral, intranasal, intravenous, intra-arterial, intramuscular, subcutaneous or intraocular administration and the like.

In particular, the compound according to the present disclosure or the pharmaceutical composition according to the present disclosure can be formulated for a topical, enteral, oral, parenteral, intranasal, intravenous, intra-arterial, intramuscular, subcutaneous or intraocular administration and the like.

Preferably, the compound according to the invention or the pharmaceutical composition according to the present disclosure is administered by enteral or parenteral route of administration. When administered parenterally, the compound according to the present disclosure or the pharmaceutical composition according to the present disclosure is preferably administered by intravenous route of administration. When administered enterally, the compound according to the present disclosure or the pharmaceutical composition according to the present disclosure is preferably administered by oral route of administration.

The pharmaceutical composition comprising the molecule is formulated in accordance with standard pharmaceutical practice (Lippincott Williams & Wilkins, 2000 and Encyclopedia of Pharmaceutical Technology, eds. J. Swarbrick and J. C. Boylan, 1988-1999, Marcel Dekker, New York) known by a person skilled in the art.

For oral administration, the composition can be formulated into conventional oral dosage forms such as tablets, capsules, powders, granules and liquid preparations such as syrups, elixirs, and concentrated drops. Nontoxic solid carriers or diluents may be used which include, for example, pharmaceutical grades of mannitol, lactose, starch, magnesium stearate, sodium saccharine, talcum, cellulose, glucose, sucrose, magnesium, carbonate, and the like. For compressed tablets, binders, which are agents which impart cohesive qualities to powdered materials, are also necessary. For example, starch, gelatine, sugars such as lactose or dextrose, and natural or synthetic gums can be used as binders. Disintegrants are also necessary in the tablets to facilitate break-up of the tablet. Disintegrants include starches, clays, celluloses, algins, gums and crosslinked polymers. Moreover, lubricants and glidants are also included in the tablets to prevent adhesion to the tablet material to surfaces in the manufacturing process and to improve the flow characteristics of the powder material during manufacture. Colloidal silicon dioxide is most commonly used as a glidant and compounds such as talc or stearic acids are most commonly used as lubricants.

For transdermal administration, the composition can be formulated into ointment, cream or gel form and appropriate penetrants or detergents could be used to facilitate permeation, such as dimethyl sulfoxide, dimethyl acetamide and dimethylformamide.

For transmucosal administration, nasal sprays, rectal or vaginal suppositories can be used. The active compound can be incorporated into any of the known suppository bases by methods known in the art. Examples of such bases include cocoa butter, polyethylene glycols (carbowaxes), polyethylene sorbitan monostearate, and mixtures of these with other compatible materials to modify the melting point or dissolution rate.

Pharmaceutical compositions according to the invention may be formulated to release the active drug substantially immediately upon administration or at any predetermined time or time period after administration.

The compound according to the invention or the pharmaceutical composition according to the present disclosure may be administered as a single dose or in multiple doses. Preferably, the treatment is administered regularly, preferably between every day and every month, more preferably between every day and every two weeks, more preferably between every day and every week, even more preferably the treatment is administered every day. In a particular embodiment, the treatment is administered daily, optionally 1, 2 or 3 times a day.

The duration of treatment with the compound according to the invention or the pharmaceutical composition according to the invention can be weeks, months or even years. In particular, the duration of treatment may last as long as the disease persists.

The amount of compound according to the present disclosure or of pharmaceutical composition according to the present disclosure to be administered has to be determined by standard procedure well known by those of ordinary skills in the art. Physiological data of the patient (e.g. age, size, and weight) and the routes of administration have to be taken into account to determine the appropriate dosage, so as a therapeutically effective amount will be administered to the patient.

In a particular aspect, the total compound dose for each administration of the compound according to the present disclosure or of the pharmaceutical composition according to the present disclosure is comprised between 0.00001 and 1 g.

The form of the pharmaceutical compositions, the route of administration and the dose of administration of the compound according to the present disclosure, or the pharmaceutical composition according to the present disclosure can be adjusted by those skilled in the art according to the type and severity of the disease, and to the patient, in particular its age, weight, sex, and general physical condition.

Further aspects and advantages of the present invention will be described in the following examples, which should be regarded as illustrative and not limiting.

### EXAMPLES

### Results

Mice were fed with a high-fat diet for 30 weeks and then treated by oral administration, twice daily with either compound #157 or once daily with OCA for 8 weeks. Effects on portal/periportal inflammation and fibrosis and on lobular inflammation were determined before treatment and upon study completion.

Compound #157 resolves portal/periportal inflammation and fibrosis following 8 weeks of treatment in a NASH mouse model (See FIGS. 1 and 2). On the opposite, as shown in FIGS. 1 and 3, OCA results in a significant anti-inflammatory effect in the lobular region (FIG. 3) but not in the portal/periportal region (FIG. 1).

### Materials and Methods

Five week old, male C₅₇BL/6JRj mice were fed either chow diet (Altromin 1324, Brogaarden, Denmark) or AMLN diet (40% total fat kcal of which 18.5% were trans-fat kcal, 20% fructose, 2% cholesterol; Research Diets #D09100301). This diet was maintained for 29 weeks prior to study initiation.

Three weeks prior to study initiation, liver biopsies were taken and animals with steatosis grade <2 and fibrosis stage <1 were deselected from the study. Prior to first administration of test article, stratified randomization of mice into treatment groups was performed according to collagen 1a1 (IHC) morphometry from the week -3 biopsies. Each treatment group consistent of 12 mice.

For a total of 8 weeks, Compound #157 was administered orally, twice daily (6 AM & 4 PM) at a concentration of 7 mg/kg or 20 mg/kg. Compound #157 was administered in a total volume of 5 ml/kg of vehicle (1.5% CMC+0.25% Tween 80 in deionized water).

For a total of 8 weeks, Obeticholic acid (OCA) was administered orally, once daily (6 AM) at a concentration of 30mg/kg. OCA was administered in a total volume of 5m1/kg of vehicle (1.5% CMC+0.25% Tween 80 in deionized water).

At termination, right medial and/or left lateral lobes of the liver (>50% of each lobe harvested) were excised and fixed in 10% neutral-buffered formalin (at least 7 days at room temperature). Liver tissue was paraffin embedded, sectioned (5 uM), and mounted taking care to select similarly sized sections representative of both the tissue edge and center. Hematoxylin and eosin stains were used for morphological analyses, and Masson's trichrome and Sirius red stains were used for assessment of hepatic fibrosis. Histopathological analysis was performed by a pathologist blinded to the study. NAFLD and NASH were scored by use of criteria outlined by Kleiner et al. Hepatology 41, p. 1313-21, 2005.

Inflammation was evaluated by counting the number of hematoxylin and eosin (H&E) positive inflammatory foci per field using a 200× magnification (min. 5 fields per animal). A focus was defined as a cluster, not a row, of >3 inflammatory cells. Acidophil bodies were not included in this assessment. Inflammation score was defined as follows:

| | |
|---|---|
| No foci | Score = 0 |
| <2 foci | Score = 1 |
| 2-4 foci | Score = 2 |
| >4 foci | Score = 3 |

Portal fibrosis was quantified using Visiopharm Histopathology Image Analysis software (Visioparm, Hørsholm, Denmark). Portal triads were annotated in Picro Sirius Red stained slides and segmentation was carried out using the convolutional neural network Deeplabv3+ (Chen, Liang-Chieh, et al. Proceedings of the European conference on computer vision (ECCV); 2018)). The periportal zone was defined as a distance of 100 μm from the portal area. Periportal fibrosis was then detected in the periportal zone using the linear Bayesian method and reported as an area fraction.

1-8. (canceled)

19. A method of treating hepatic portal/periportal inflammation in a subject comprising the administration of a compound of formula (I) to said subject, 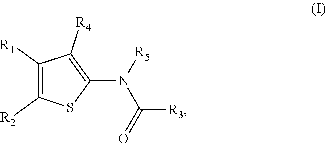
wherein:
R₁ represents a fused arylcycloalkyl or a cycloalkyl optionally substituted by at least one radical selected from the group consisting of a halogen, a (C₁-C₆)alkyl or a (C₁-C₆)alkyloxy optionally substituted by at least one halogen, a hydroxy, a —CO—R₆ or a —CO₂R₆ with R₆ being a hydrogen or a (C₁-C₆)alkyl, and an optionally substituted aryl;
R₂ is a hydrogen, a halogen, a (C₁-C₆)alkyl optionally substituted by at least one halogen, an optionally substituted aryl, or an optionally substituted cycloalkyl;
R₃ represents:
a 5-10 membered ring, saturated or unsaturated selected from the group consisting of an aryl optionally fused to a heterocycloalkyl, a dioxole, a morpholine, a dioxane, a tetrahydropyran, or a tetrahydrofuran, a heteroaryl, a cycloalkyl, a heterocycloalkyl, and a 5-10 membered bridged carbocyclyl or heterocyclyl,
said 5-10 membered ring is optionally substituted by at least one radical selected from the group consisting of a halogen, a (C₁-C₆)alkyl optionally substituted by at least one halogen or by an optionally bridged heterocycloalkyl optionally substituted by a (C₁-C₆)alkyl,
a —NH-(C₁-C₆)alkyl or a —N—((C₁-C₆)alkyl)₂, optionally substituted by at least one radical selected from the group consisting of a heterocycloalkyl,
a cycloalkyl, a hydroxyl, a thi acycl oalkyl-1,1di oxide and a (C₁-C₆)alkyloxy,
a —NH-heterocycloalkyl, a —NH-cycloalkyl, a —N((C₁-C₆)alkyl)-heterocycloalkyl, or a —NH((C₁-C₆)alkyl)-thiacycloalkyl-1,1dioxide, optionally substituted by a hydroxyl, a (C₁-C₆)alkyl, a (C₁-C₆)alkyloxy or a —CO—R₆ with R₆ being a hydrogen or a (C₁-C₆)alkyl,
a hydroxy, a —CN, a —CO—R₆ or a —CO₂R₆ with R₆ being a hydrogen or a (C₁-C₆)alkyl,
a (C₁-C₆)alkyloxy optionally substituted by at least one radical selected from the group consisting of a halogen, a hydroxy, a (C₁-C₆)alkyloxy, a —NR₇R₈ with R₇ and Rs are independently a hydrogen or a (C₁-C₆)alkyl, a —NHCOR₉, a —NHCO₂R₉, with R₉ being a (C₁-C₆)alkyl, a —CO₂R₆ with R₆ being a hydrogen or a (C₁-C₆)alkyl, and a heterocycle,
a —NHCOR₉, a —NHCO₂R₉, or a —SO2R₉, with R₉ being a (C₁-C₆)alkyl, and a heterocycloalkyl, a bridged heterocycloalkyl, a heterocycloalkyloxy, a cycloalkyloxy, a thiaheterocycloalkyl-1,1-dioxide or a spiroheterocycloalkyl, optionally substituted by a (C₁-C₆)alkyl, a (C₁-C₆)alkyloxy, a hydroxy, a ketone, a halogen or a (C₁-C₆)alkyl optionally substituted by a (C₁-C₆)alkyloxy, or
a (C₁-C₆)alkyl or a (C₂-C₆)alkenyl, optionally substituted by a 5-10 membered ring as defined above or a —CO₂R₆ with R₆ being a hydrogen or a (C₁-C₆)alkyl;
R₄ represents a —COOH;
R₅ represents:
a hydrogen, or a (C₁-C₆)alkyl optionally substituted by at least one halogen; and the stereoisomers, and the pharmaceutical salts thereof.

20. The method according to claim 19, wherein the subject has hepatic portal/periportal inflammation of grade 1 or grade 2 as defined in the specification.

21. The method according to claim 19, wherein the subject suffers from a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury, biliary atresia, idiopathic neonatal hepatitis syndrome, progressive familial intrahepatic cholestasis (PFIC) and primary and secondary mitochondrial hepatopathies.

22. The method according to claim 19, wherein the subject suffers from a disease selected from the group consisting of pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), biliary atresia, idiopathic neonatal hepatitis syndrome, progressive familial intrahepatic cholestasis (PFIC), Reye's syndrome, Indian childhood cirrhosis, Idiopathic infantile copper toxicosis, Neonatal iron storage disease, Type I Tyrosinemia, and Zellweger syndrome.

23. The method according to claim 19, wherein the subject has a hepatic portal/periportal inflammation, and, optionally a hepatic portal/periportal fibrosis, and suffers from a disease selected from the group consisting of viral hepatitis, chronic viral hepatitis, hemochromatosis and NASH.

24. The method according to claim 19, wherein the disease is selected from the group consisting of pediatric non-alcoholic fatty liver disease (NAFLD) and pediatric non-alcoholic steatohepatitis (NASH).

25. The method according to claim 19, wherein Ri represents an optionally substituted fused arylcycloalkyl.

26. The method according to claim 19, wherein Ri represents an optionally substituted fused arylcycloalkyl selected from the group consisting of an indanyl, a 1,2,3,4-tetrahydronaphtalenyl, and a 6,7,8,9-tetrahydro-5H-benzo[7]annulenyl

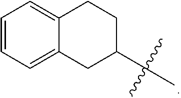 [SMILES: *C1CCc2ccccc2C1]

27. The method according to claim 19, wherein R₃ represents an aryl optionally fused to a heterocycloalkyl, a dioxole, a morpholine, a dioxane, a tetrahydropyran, and a tetrahydrofuran, or a heteroaryl, said aryl, fused aryl, or heteroaryl being optionally substituted by at least one radical selected from the group consisting of:
a heterocycloalkyl or a bridged heterocycloalkyl, optionally substituted by a (C₁-C₆)alkyl, a (C₁-C₆)alkyloxy, or a ketone;
a thiaheterocycloalkyl-1,1-dioxide, a heterocycloalkyloxy, or a cycloalkyloxy;
a (C₁-C₆)alkyloxy or a (C₁-C₆)alkyl, optionally substituted by at least one halogen or a (C₁-C₆)alkyloxy;
a halogen;
a —NH-(C₁-C₆)alkyl or a —N—((C₁-C₆)alkyl)₂, optionally substituted by a heterocycloalkyl, a cycloalkyl, a hydroxyl, a thiacycloalkyl-1,1-dioxide or a (C₁-C₆)alkyloxy;
a —NH-heterocycloalkyl, a —N((C₁-C₆)alkyl)-heterocycloalkyl, or a —NH((C₁-C₆)alkyl)-thiacycloalkyl-1,1-dioxide;
a hydroxy;
a —CN;
a (C₁-C₆)alkyl substituted by an optionally bridged heterocycloalkyl or an optionally substituted heterocycloalkyl; and
a —SO2R₉, with R₉ being a (C₁-C₆)alkyl.

28. The method according to claim 19, wherein R₃ represents a pyridinyl, a pyrimidinyl, or a phenyl, optionally substituted by at least one radical selected from the group consisting of:
a morpholinyl optionally substituted by at least one methyl;
a —NH-tetrahydropyranyl;
a —NH-(C₁-C₆)alkyl or a —N(CH₃)(C₁-C₆)alkyl), optionally substituted by a tetrahydropyranyl, a cyclohexyl, an optionally bridged morpholinyl optionally substituted by at least one methyl, a thiacycloalkyl-1,1-dioxide, a hydroxy, or a (C₁-C₆)alkyloxy;
an azetidinyl optionally substituted by a (C₁-C₆)alkyloxy;
a pyrrolidin-2-one;
a 6-oxa-3-azabicyclo[3.1.1]heptane, or a 8 oxa-3-azabicyclo[3.2.1]octane;
a (C₁-C₆)alkyloxy, optionally substituted by at least one halogen or one (C₁-C₆)alkyloxy, a halogen;
a hydroxy;
a —CN;
a —SO₂—CH₃;
a 1,1-dioxo-1,2-thiazolidin;
a cyclobutyloxy, or a tetrahydropyranyloxy;
a (C₁-C₆)alkyl optionally substituted by at least one halogen; and
a (C₁-C₆)alkyl substituted by a morpholinyl optionally substituted by at least one methyl, a 6-oxa-3-azabicyclo[3.1.1]heptane, a 8 oxa-3-azabicyclo[3.2.1]octane or a tetrahydropyranyl.

29. The method according to claim 19, wherein R₂ represents a hydrogen, a halogen, or an optionally substituted (C₃-C₆)cycloalkyl.

30. The method according to claim 19, wherein said compound is selected from the group consisting of compounds of the table A.

31. The method according to claim 19, wherein said compound is selected from the group consisting of compounds of the table B.

32. The method according to claim 19, wherein R₁ is 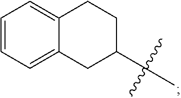 [SMILES: *C1CCc2ccccc2C1]
R₂ is a hydrogen or a halogen; R₃ is a phenyl optionally substituted by a halogen; and Rs is a hydrogen.

33. The method according to claim 19, wherein the compound is administered in combination with another active ingredient.

34. The method according to claim 33, wherein the other active ingredient is Obeticholic acid.

35. A pharmaceutical composition comprising a compound according to claim 19 and Obeticholic acid.

36. A method of treating a subject having a disease comprising the administration of a composition according to claim 35 to the subject, said disease being pediatric non-alcoholic fatty liver disease (NAFLD), pediatric non-alcoholic steatohepatitis (NASH), autoimmune hepatitis, primary biliary cholangitis (PBC), primary sclerosing cholangitis (PSC), Chronic Chemical and Drug Induced Liver Injury, biliary atresia, idiopathic neonatal hepatitis syndrome, progressive familial intrahepatic cholestasis (PFIC) and primary and secondary mitochondrial hepatopathies; or a disease characterized by the occurrence of hepatic portal/periportal inflammation, optionally with a hepatic portal/periportal fibrosis selected from the group consisting of viral hepatitis, chronic viral hepatitis, hemochromatosis and NASH.
